(19) United States
(12) Patent Application Publication
(10) Pub. No.: US 2007/0293553 A1
(43) Pub. Date: Dec. 20, 2007

(54) Diphenylurea Derivatives Useful As Potassium Channel Activators

(76) Inventors: Bjarne H. Dahl, Lynge (DK); Palle Christophersen, Ballerup (DK); Joachim Demnitz, Kobenhavn (DK)

Correspondence Address: BIRCH STEWART KOLASCH & BIRCH, PO BOX 747, FALLS CHURCH, VA, 22040-0747, US

(21) Appl. No.: 11/791,340

(22) PCT Filed: Dec. 14, 2005
(86) PCT No.: PCT/EP05/56766

§ 371 Date: May 23, 2007

Related U.S. Application Data
(60) Provisional application No. 60/637,775, filed on Dec. 22, 2004.

(30) Foreign Application Priority Data
Dec. 17, 2004 (DK) PA 2004 01953

(51) Int. Cl. A61K 31/41; A61P 11/00; A61P 13/00; A61P 25/00

(52) U.S. Cl. 514/381

(57) ABSTRACT

The present invention relates to the medical use of a certain group of diphenyl urea derivatives as potassium channel blockers for treating cardiovascular diseases, an obstructive or inflammatory airway disease, urinary incontinence, psychosis, epilepsy or pain, or for facilitating the blood-brain barrier permeability for other therapeutic substances. In other aspects the invention relates to the use of these compounds in a method of therapy.

### TECHNICAL FIELD

The present invention relates to the medical use of a certain group of diphenyl urea derivatives as potassium channel blockers for treating cardiovascular diseases, an obstructive or inflammatory airway disease, urinary incontinence, psychosis, epilepsy or pain, or for facilitating the blood-brain barrier permeability for other therapeutic substances. In other aspects the invention relates to the use of these compounds in methods of therapy.

### BACKGROUND ART

Ion channels are transmembrane proteins, which catalyse the transport of inorganic ions across cell membranes. The ion channels participate in processes as diverse as the generation and timing of action potentials, synaptic transmissions, secretion of hormones, contraction of muscles, etc.

Many drugs exert their effects via modulation of ion channels. Examples are anti-epileptic compounds like Phenyloin and Lamotrigine, which block voltage dependent Na⁺-channels in the brain, anti-hypertensive drugs like Nifedipine and Diltiazem, which block voltage dependent Ca²⁺-channels in smooth muscle cells, and stimulators of insulin release like Glibenclamide and Tolbutamide, which block an ATP-regulated K⁺-channel in the pancreas.

Potassium Channels and Potassium Channel Modulators

All mammalian cells express potassium (K⁺) channels in their cell membranes, and the channels play a dominant role in the regulation of the membrane potential. In nerve and muscle cells they regulate the frequency and form of the action potential, the release of neurotransmitters, and the degree of broncho- and vasodilation.

From a molecular point of view, the K⁺ channels represent the largest and most diverse group of ion channels. For an overview they can be divided into five large subfamilies: Voltage-activated K⁺ channels (Kᵥ), long QT related K⁺ channels (KvLQT), inward rectifiers (K_{IR}), two-pore K⁺ channels (K_{TP}), and calcium-activated K⁺ channels (K_{ca}).

The latter group, the Ca²⁺-activated K⁺ channels, consists of three well-defined subtypes: SK channels, IK channels and BK channels. SK, IK and BK refer to the single-channel conductance (Small, Intermediate and Big conductance K channel). The SK, IK, and BK channels exhibit differences in e.g. voltage- and calcium-sensitivity, pharmacology, distribution and function.

WO 94/22807 describes diphenyl urea derivatives useful as BK potassium channel modulators for the manufacture of a medicament for the treatment of arterial hypertension, coronary artery spasms, asthma, irritable bowl syndrome, spastic bladder, ischemia, psychosis and convulsions. However, the use of such compounds for treating obstructive or inflammatory airway diseases or for facilitating the blood-brain barrier permeability of therapeutic substances has never been suggested.

WO 00/01676, WO 01/02406 and WO 04/002962 describe potassium channel modulators useful for treating i.a. obstructive or inflammatory airway diseases and for facilitating the blood-brain barrier permeability of therapeutic substances. However, the use of diphenyl urea derivatives as potassium channel openings agents has never been suggested.

Chloride Channels and Chloride Channel Modulators

Chloride channels serve a wide variety of specific cellular functions and contribute to the normal function of i.a. skeletal and smooth muscle cells. Chloride channels are probably found in every cell, from bacteria to mammals. Their physiological tasks range from cell volume regulation to stabilization of the membrane potential, transepithelial or transcellular transport and acidification of intracellular organelles.

Chloride channels are currently believed to be encoded by at least four gene families: the voltage-gated chloride channels (CIC), the ligand-gated chloride channels (Glycin and GABA receptors), the CFTR, and the calcium-activated chloride channels (CICa).

CIC channels, members of a large family of voltage gated chloride channels, are found throughout biology in prokaryotic and eukaryotic cells. The nine isoforms of CIC channels in humans reside in the plasma membrane and in the membrane of intracellular organelles. They are involved in such important processes as electrical signalling in muscle and certain neurons, transepithelial flux of electrolytes in the kidney and acidification of intracellular vesicles.

WO 98/47879 and WO 00/24707 describe phenyl derivatives which are potent chloride channel blockers and as such useful in the treatment of diseases and conditions responding to blockade of chloride channels, such as sickle cell anemia, brain oedema following ischaemia or tumours, diarrhea, hypertension (diuretic), osteoporosis, and for the reduction of the intraocular pressure for the treatment of disorders such as glaucoma, for the treatment of allergic and inflammatory conditions and for the promotion of wound healing. However, the use of these compounds for treating obstructive or inflammatory airway diseases or for facilitating the blood-brain barrier permeability of therapeutic substances has never been suggested.

### SUMMARY OF THE INVENTION

According to the present invention it has now been found that a particular group of diphenyl urea derivatives, formerly believed to be blockers of chloride channels, also possesses valuable therapeutic properties as activators of potassium channels. Moreover it has been found that this group of diphenyl urea derivatives are particularly useful for treating cardiovascular diseases, obstructive or inflammatory airway diseases, urinary incontinence, psychosis, epilepsy or pain, or for facilitating the blood-brain barrier permeability for other therapeutic substances.

Therefore, in its first aspect, the invention relates to the medical utility of a particular group of diphenyl urea derivatives, namely the use of these compounds as potassium channel activators. More specifically the invention relates to the use of a particular group of diphenyl urea derivatives for treating cardiovascular diseases, obstructive or inflammatory airway diseases, urinary incontinence, psychosis, epilepsy or pain, or for facilitating the blood-brain barrier permeability for other therapeutic substances.

The diphenyl urea derivatives for use according to the invention may be characterised by Formula I

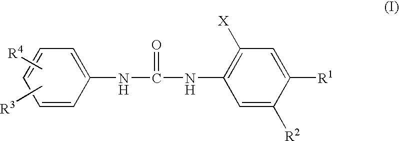

or a pharmaceutically acceptable salt thereof, wherein X represents hydroxy, carboxy, a tetrazolyl group, an oxadiazolyl group or a triazolyl group; R¹ represents hydrogen, alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, N-alkyl-piperazinyl-carbonyl, carbamoyl-N-alkyl-piperazine, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride, phenyl, naphthyl, pyridyl, furyl or thienyl; or R¹ represents phenyl substituted once or twice with alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, carbamoyl-N-alkyl-piperazine, N-alkyl-piperazinyl-carbonyl, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride; R² represents hydrogen, halo, haloalkyl, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, haloalkoxy, carboxy, alkyl-carbonyl, alkoxy-carbonyl, amino-carbonyl, benzoyl, acetyl, phenyl, pyridyl; or phenyl substituted with alkyl, halo or haloalkyl; and R⁴ represents hydrogen, alkyl, halo, haloalkyl, nitro, hydroxy, alkoxy, phenyl, pyridyl, or phenyl substituted with haloalkyl; or R³ and R⁴ together with the phenyl to which they are attached form a naphthyl group.

In another aspect the invention relates to methods of treatment, prevention or alleviation of an obstructive or inflammatory airway disease, urinary incontinence, psychosis, epilepsy or pain in a living animal body, including a human, which method comprises the step of administering to such a living animal body in need thereof, a therapeutically effective amount of the diphenyl urea derivative according to the invention, or a pharmaceutically-acceptable salt thereof.

In yet another aspect the invention relates to methods of increasing the blood-brain barrier permeability in a living animal body, including a human, which method comprises the step of administering to such a living animal body in need thereof, a therapeutically effective amount of the diphenyl urea derivative according to the invention, or a pharmaceutically-acceptable salt thereof.

Other objects of the invention will be apparent to the person skilled in the art from the following detailed description and examples.

### DETAILED DISCLOSURE OF THE INVENTION

Diphenyl Urea Derivatives

The diphenyl urea derivatives for use according to the invention may be characterised by Formula I

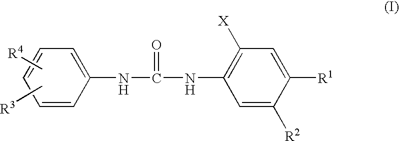

or a pharmaceutically acceptable salt thereof, wherein X represents hydroxy, carboxy, a tetrazolyl group, an oxadiazolyl group or a triazolyl group; R¹ represents hydrogen, alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, N-alkyl-piperazinyl-carbonyl, carbamoyl-N-alkyl-piperazine, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride, phenyl, naphthyl, pyridyl, furyl or thienyl; or R¹ represents phenyl substituted once or twice with alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, carbamoyl-N-alkyl-piperazine, N-alkyl-piperazinyl-carbonyl, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride; R² represents hydrogen, halo, haloalkyl, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, haloalkoxy, carboxy, alkyl-carbonyl, alkoxy-carbonyl, amino-carbonyl, benzoyl, acetyl, phenyl, pyridyl; or phenyl substituted with alkyl, halo or haloalkyl; and R⁴ represents hydrogen, alkyl, halo, haloalkyl, nitro, hydroxy, alkoxy, phenyl, pyridyl, or phenyl substituted with haloalkyl; or R³ and R⁴ together with the phenyl to which they are attached form a naphthyl group.

In a preferred embodiment of the invention X represents hydroxy, carboxy, a tetrazolyl group, an oxadiazolyl group or a triazolyl group.

In a more preferred embodiment X represents hydroxy, carboxy, 1H-tetrazol-5-yl, 5-oxo-4,5-dihydro-[1,2,4]oxadiazol-3-yl, 4-hydroxy-1,2,4-triazol-3-yl or 3-oxo-1,2-dihydro-1,2,4-triazol-1-yl.

In an even more preferred embodiment X represents carboxy, 1H-tetrazol-5-yl, 5-oxo-4,5-dihydro-[1,2,4]oxadiazol-3-yl, 2-oxo-3H-1,3,4-oxadiazol-5-yl, 4-hydroxy-1,2,4-triazol-3-yl or 3-oxo-1,2-dihydro-1,2,4-triazol-1-yl.

In a yet more preferred embodiment X represents a tetrazolyl group, an oxadiazolyl group or a triazolyl group.

In a still more preferred embodiment X represents 1H-tetrazol-5-yl, 5-oxo-4,5-dihydro-[1,2,4]oxadiazol-3-yl, 2-oxo-3H-1,3,4-oxadiazol-5-yl, 4-hydroxy-1,2,4-triazol-3-yl or 3-oxo-1,2-dihydro-1,2,4-triazol-1-yl.

In another preferred embodiment of the invention R¹ represents hydrogen, alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, N-alkyl-piperazinyl-carbonyl, carbamoyl-N-alkyl-piperazine, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride, phenyl, naphthyl, pyridyl, furyl or thienyl; or R¹ represents phenyl substituted once or twice with alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, carbamoyl-N-alkyl-piperazine, N-alkyl-piperazinyl-carbonyl, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride.

In a more preferred embodiment R¹ represents hydrogen, methyl, ethyl, chloro, fluoro, bromo, trifluoromethyl, hydroxy, methoxy, ethoxy, trifluoromethoxy, nitro, cyano, amino, N-phenyl-amino, N-benzoyl-amino, methyl-carbonyl-amino, carboxy, acetyl, ethyl-carbonyl, cyclopropyl-carbonyl, methoxy-carbonyl, ethoxy-carbonyl, carbamoyl, N-methyl-carbamoyl, N,N-dimethyl-carbamoyl, N-phenyl-carbamoyl, N,N-dimethyl-carbamoyl, N,N-diethyl-carbamoyl, N-methyl-N-acetic acid carbamoyl, anilino-carbonyl, pyrrolidinyl-carbonyl, piperidinyl-carbonyl, piperazinyl-carbonyl, N-methyl-piperazinyl-carbonyl, N,N-dimethyl acryl-amide, amino-carbonyl-methyl, N-methyl-amino-carbonyl-methyl, N,N-dimethyl-propionamide, N,N-dimethyl acryl-amide, sulfamoyl, N-methyl-sulfamoyl, N,N-dimethylsulfamoyl, sulfonamido-N-methyl-piperazinium chloride, phenyl, 1-naphthyl, 2-naphthyl, 3-thienyl or 3-pyridyl; or R¹ represents phenyl substituted once or twice with methyl, ethyl, chloro, fluoro, bromo, trifluoromethyl, hydroxy, methoxy, ethoxy, trifluoromethoxy, nitro, cyano, amino, N-phenyl-amino, N-benzoyl-amino, methyl-carbonyl-amino, carboxy, acetyl, ethyl-carbonyl, cyclopropyl-carbonyl, methoxy-carbonyl, ethoxy-carbonyl, carbamoyl, N-methyl-carbamoyl, N,N-dimethyl-carbamoyl, N-phenyl-carbamoyl, N,N-dimethyl-carbamoyl, N,N-diethyl-carbamoyl, N-methyl-N-acetic acid carbamoyl, anilino-carbonyl, pyrrolidinyl-carbonyl, piperidinyl-carbonyl, piperazinyl-carbonyl, N-methyl-piperazinyl-carbonyl, N,N-dimethyl acryl-amide, amino-carbonyl-methyl, N-methyl-amino-carbonyl-methyl, N,N-dimethyl-propionamide, N,N-dimethyl acryl-amide, sulfamoyl, N-methyl-sulfamoyl, N,N-dimethylsulfamoyl and/or sulfonamido-N-methyl-piperazinium chloride.

In an even more preferred embodiment R¹ represents hydrogen, alkyl, halo, haloalkyl, hydroxy, alkoxy, haloalkoxy, nitro, amino, alkyl-carbonyl-amino, N-phenyl-amino, N-benzoyl-amino, N,N-dialkyl acryl-amide, N,N-dialkyl-amino-carbonyl, N,N-dialkyl-amino-carbonyl-alkyl, alkoxy-carbonyl, phenyl, naphthyl, pyridyl, furyl or thienyl; or R¹ represents phenyl substituted with alkyl, halo, haloalkyl, haloalkoxy, nitro, amino, carboxy, alkoxy-carbonyl, amino-carbonyl (carbamoyl), N,N-dialkyl-amino-carbonyl, N,N-dialkyl-amino-carbonyl-alkyl, N-phenyl-amino-carbonyl, N-alkyl-N-acetic acid amino-carbonyl, N-acetic acid-amino-carbonyl, anilino-carbonyl, piperidinyl-carbonyl, carbamoyl-N-alkyl-piperazine, N,N-dialkyl-sulfamoyl or sulfonamido-N-alkyl-piperazinium chloride.

In a yet more preferred embodiment R¹ represents hydrogen, methyl, ethyl, chloro, fluoro, bromo, trifluoromethyl, hydroxy, methoxy, ethoxy, trifluoromethoxy, nitro, amino, methyl-carbonyl-amino, N-phenyl-amino, N-benzoyl-amino, N,N-dimethyl acryl-amide, N,N-dimethyl-amino-carbonyl, N,N-dimethyl-amino-carbonyl-ethyl, methoxy-carbonyl, phenyl, 1-naphthyl, 2-naphthyl, 3-pyridyl, 3-furyl or 3-thienyl; or R¹ represents phenyl substituted with methyl, ethyl, chloro, fluoro, bromo, trifluoromethyl, trifluoromethoxy, nitro, amino, carboxy, methoxy-carbonyl, amino-carbonyl (carbamoyl), N,N-dimethyl-amino-carbonyl, N,N-dimethyl-amino-carbonyl-ethyl, N-phenyl-amino-carbonyl, N-methyl-N-acetic acid amino-carbonyl, N-acetic acid-amino-carbonyl, anilino-carbonyl, piperidinyl-carbonyl, carbamoyl-N-alkyl-piperazine, N,N-dimethyl-sulfamoyl or sulfonamido-N-methyl-piperazinium chloride.

In a third preferred embodiment of the invention R² represents hydrogen, halo, haloalkyl, alkoxy, alkoxy-carbonyl, nitro, halophenyl, haloalkyl-phenyl or haloalkoxy-phenyl.

In a more preferred embodiment R² represents hydrogen, chloro, fluoro, bromo, trifluoromethyl, methoxy, ethoxy, acetyl, nitro, chloro-phenyl, fluorophenyl, trifluoromethyl-phenyl or trifluoromethoxy-phenyl.

In a fourth preferred embodiment of the invention R³ represents hydrogen, alkyl, halo, haloalkyl, nitro, hydroxy, alkoxy, haloalkoxy, carboxy, alkyl-carbonyl, alkoxy-carbonyl, amino-carbonyl, benzoyl, phenyl, pyridyl, or phenyl substituted with alkyl, halo, haloalkyl or haloalkoxy; and R⁴ represents hydrogen, alkyl, halo, haloalkyl, nitro, hydroxy, alkoxy, haloalkoxy, carboxy, alkyl-carbonyl, alkoxy-carbonyl, amino-carbonyl, benzoyl, phenyl, pyridyl, or phenyl substituted with alkyl, halo, haloalkyl or haloalkoxy; or R³ and R⁴ together with the phenyl to which they are attached form a naphthyl group.

In a more preferred embodiment R³ represents hydrogen, methyl, ethyl, chloro, fluoro, bromo, trifluoromethyl, nitro, hydroxy, methoxy, ethoxy, trifluoromethoxy, carboxy, acetyl, methoxy-carbonyl, ethoxy-carbonyl, carbamoyl, benzoyl, phenyl or 3-pyridyl, or phenyl substituted with methyl, ethyl, chloro, fluoro, bromo, trifluoromethyl or trifluoromethoxy.

In another preferred embodiment R⁴ represents hydrogen, methyl, ethyl, chloro, fluoro, bromo, trifluoromethyl, nitro, hydroxy, methoxy, ethoxy, trifluoromethoxy, carboxy, acetyl, methoxy-carbonyl, ethoxy-carbonyl, carbamoyl, benzoyl, phenyl or 3-pyridyl, or phenyl substituted with methyl, ethyl, chloro, fluoro, bromo, trifluoromethyl or trifluoromethoxy.

In a fifth preferred embodiment the diphenyl urea derivative for use according to the invention is represented by Formula II

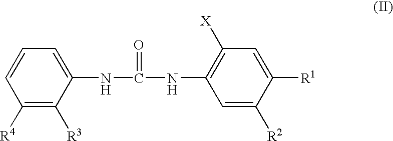

or a pharmaceutically acceptable salt thereof, wherein X, R¹ and R² are as defined above; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl or pyridyl; or phenyl substituted with alkyl, halo or haloalkyl; and R⁴ represents hydrogen, alkyl, halo, haloalkyl, nitro, alkoxy, or phenyl; or phenyl substituted with haloalkyl; or R³ and R⁴ together with the phenyl to which they are attached form a naphthyl group.

In a preferred embodiment X represents a tetrazolyl group, an oxadiazolyl group or a triazolyl group; R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, piperidin-1-yl-carbonyl, amino-carbonyl-N-alkyl-piperazine, N,N-dialkylsulfamoyl or sulfonamido-N-alkyl-piperazinium chloride; and R² represents hydrogen, halo, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen; alkyl; halo; haloalkyl; nitro; alkoxy; phenyl or phenyl substituted with haloalkyl; or R³ and R⁴ together with the phenyl to which they are attached form a naphthyl group.

In a more preferred embodiment X represents a tetrazolyl group, an oxadiazolyl group or a triazolyl group; R¹ represents hydrogen, halo, haloalkyl, nitro, piperidin-1-yl-carbonyl-phenyl or N,N-dialkyl-amino-carbonyl-phenyl; R² represents hydrogen; R³ represents alkyl, halo, haloalkyl, nitro, hydroxy, alkoxy, phenyl; phenyl substituted with alkyl, halo or haloalkyl; and R⁴ represents hydrogen, alkyl, halo, haloalkyl or nitro; or R³ and R⁴ together with the phenyl to which they are attached form a naphthyl group.

In an even more preferred embodiment X represents tetrazolyl; R¹ represents halo, piperidin-1-yl-carbonyl-phenyl or N,N-dialkyl-amino-carbonyl-phenyl; R² represents hydrogen; and R³ represents alkyl, halo, haloalkyl, nitro, 4-alkyl-phenyl, 4-halophenyl or 4-haloalkyl-phenyl; and R⁴ represents hydrogen; or R³ and R⁴ together with the phenyl to which they are attached form a naphthyl group.

In a yet more preferred embodiment X represents 1H-tetrazol-5-yl; R¹ represents bromo, piperidin-1-yl-carbonyl-phenyl or N,N-dimethyl-amino-carbonyl-phenyl; R² represents hydrogen; and R³ represents methyl, ethyl, chloro, fluoro, bromo, trifluoromethyl, nitro, 4-methyl-phenyl, 4-trifluoromethyl, 4-chlorophenyl or 4-fluorophenyl; and R⁴ represents hydrogen; or R³ and R⁴ together with the phenyl to which they are attached form a naphthyl group.

In most preferred embodiment the diphenyl urea derivative for use according to the invention is
N-(2-Nitrophenyl)-N′-[4-bromo-2-(1H-tetrazol-5-yl)phenyl]urea;
N-(2-Methylphenyl)-N′-[4-bromo-2-(1H-tetrazol-5-yl)phenyl]urea;
N-(2-Ethylphenyl)-N′-[4-bromo-2-(1H-tetrazol-5-yl)phenyl]urea;
N-(2-Trifluoromethylphenyl)-N′-[4-bromo-2-(1H-tetrazol-5-yl)phenyl]urea;
N-(2-Bromophenyl)-N′-[4-bromo-2-(1H-tetrazol-5-yl)phenyl]urea;
N-(2-Chlorophenyl)-N′-[4-bromo-2-(1H-tetrazol-5-yl)phenyl]urea;
N-(2-Fluorophenyl)-N′-[4-bromo-2-(1H-tetrazol-5-yl)phenyl]urea;
N-(2-Trifluoromethyl-phenyl)-N′-[3-(1H-tetrazol-5-yl)-4′-trifluoromethyl-biphenyl-4-yl]-urea;
N-(2-Trifluoromethyl-phenyl)-N′-[4′-fluoro-3-(1H-tetrazol-5-yl)-biphenyl-4-yl]-urea;
N-(2-Bromo-phenyl)-N′-[3-(1H-tetrazol-5-yl)-4′-trifluoromethyl-biphenyl-4-yl]-urea;
N-(2-Trifluoromethyl-phenyl)-N′-[4′-chloro-3-(1H-tetrazol-5-yl)-biphenyl-4-yl]-urea;
N-(2-Bromo-phenyl)-N′-[4′-chloro-3-(1H-tetrazol-5-yl)-biphenyl-4-yl]-urea;
N-(2-Bromo-phenyl)-N′-[4′-fluoro-3-(1H-tetrazol-5-yl)-biphenyl-4-yl]-urea;
N-(2-Fluoro-phenyl)-N′-[3-(1H-tetrazol-5-yl)-4′-trifluoromethyl-biphenyl-4-yl]-urea;
N-(2-Fluoro-phenyl)-N′-[4′-chloro-3-(1H-tetrazol-5-yl)-biphenyl-4-yl]-urea;
N-(2-Fluoro-phenyl)-N′-[4′-fluoro-3-(1H-tetrazol-5-yl)-biphenyl-4-yl]-urea;
N-(2-Fluoro-phenyl)-N′-[4′-methyl-3-(1H-tetrazol-5-yl)-biphenyl-4-yl]-urea;
N-(2-Chloro-phenyl)-N′-[4′-methyl-3-(1H-tetrazol-5-yl)-biphenyl-4-yl]-urea;
N-(2-Bromo-phenyl)-N′-[4′-methyl-3-(1H-tetrazol-5-yl)-biphenyl-4-yl]-urea;
N-(2-Trifluoromethyl-phenyl)-N′-[4′-methyl-3-(1H-tetrazol-5-yl)-biphenyl-4-yl]-urea;
N-(2-Chloro-phenyl)-N′-[3-(1H-tetrazol-5-yl)-4′-trifluoromethyl-biphenyl-4-yl]-urea;
N-(2-Chloro-phenyl)-N′-[4′-chloro-3-(1H-tetrazol-5-yl)-biphenyl-4-yl]-urea;
N-(2-Chloro-phenyl)-N′-[4′-fluoro-3-(1H-tetrazol-5-yl)-biphenyl-4-yl]-urea;
N-(2-Chloro-phenyl)-N′-[4′-(piperidin-1-yl-carbonyl)-3-(1H-tetrazol-5-yl)-biphenyl-4-yl]urea;
N-(2-Trifluoromethyl)-N′-[4′-(N″,N″-dimethyl-amino-carbonyl)-3-(1H-tetrazol-5-yl)-biphenyl-4-yl]urea;
N-(1-Naphthyl)-N′-[4-bromo-2-(1H-tetrazol-5-yl)phenyl]urea;
N-(1-Naphthyl)-N′-[4′-(N″,N″-dimethyl-amino-carbonyl)-3-(1H-tetrazol-5-yl)-biphenyl-4-yl]urea;

or a pharmaceutically acceptable salt thereof.

In a sixth preferred embodiment the diphenyl urea derivative for use according to the invention is represented by Formula III

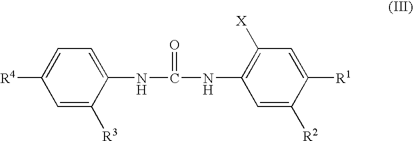

or a pharmaceutically acceptable salt thereof, wherein X, R¹ and R² are as defined above, and R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy, phenyl or phenyl substituted with haloalkyl.

In a preferred embodiment X represents a tetrazolyl group, an oxadiazolyl group or a triazolyl group; R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and R² represents hydrogen, halo, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy, phenyl or phenyl substituted with haloalkyl.

In a more preferred embodiment X represents a tetrazolyl group, an oxadiazolyl group or a triazolyl group; R¹ represents hydrogen; halo; haloalkyl; nitro; phenyl; or phenyl substituted with haloalkyl or N,N-dialkyl-amino-carbonyl; R² represents hydrogen or halo; R³ represents hydrogen, alkyl, halo, haloalkyl, nitro, hydroxy or alkoxy; and R⁴ represents hydrogen, alkyl, halo, haloalkyl, nitro, alkoxy, phenyl or phenyl substituted with haloalkyl.

In an even more preferred embodiment X represents tetrazolyl; R¹ represents hydrogen, halo, 4-haloalkyl-phenyl or N,N-dialkyl-amino-carbonyl-phenyl; R² represents hydrogen or halo; R³ represents hydrogen or halo; and R⁴ represents alkyl, halo, haloalkyl, alkoxy, nitro, phenyl or 4-haloalkyl-phenyl.

In a yet more preferred embodiment X represents 1H-tetrazol-5-yl; R¹ represents hydrogen, bromo, 4-trifluoromethyl-phenyl or N,N-dimethyl-amino-carbonyl-phenyl; R² represents hydrogen or chloro; R³ represents hydrogen or chloro; and R⁴ represents methyl, 2-propyl, chloro, bromo, trifluoromethyl, methoxy, ethoxy, nitro, phenyl or 4-trifluoromethyl-phenyl.

In a most preferred embodiment the diphenyl urea derivative for use according to the invention is
N-(2-Chloro-4-trifluoromethylphenyl)-1-[4-bromo-2-(1H-tetrazol-5-yl)phenyl]urea;
N-(4-Biphenyl)-N′-(2-(1H-tetrazol-5-yl)phenyl)urea;
N-(4-Biphenyl)-N′-(5-chloro-2-(1H-tetrazol-5-yl)phenyl)urea;
N-(4-Trifluoromethylphenyl-N′-[4-bromo-2-(1H-tetrazol-5-yl)phenyl]urea;
N-(4-Bromophenyl)-N′-[4-bromo-2-(1H-tetrazol-5-yl)phenyl]urea;
N-(4-Methylphenyl)-N′-[4-bromo-2-(1H-tetrazol-5-yl)phenyl]urea;
N-(4-[2-Propyl]phenyl)-N′-[4-bromo-2-(1H-tetrazol-5-yl)phenyl]urea;
N-(4-Methoxyphenyl)-N′-[4-bromo-2-(1H-tetrazol-5-yl)phenyl]urea;
N-(4-Ethoxyphenyl)-N′-[4-bromo-2-(1H-tetrazol-5-yl)phenyl]urea;
N-(4-Nitrophenyl)-N′-[4-bromo-2-(1H-tetrazol-5-yl)phenyl]urea;
N-(4-Chloro-phenyl)-N′-[3-(1H-tetrazol-5-yl)-4′-trifluoromethyl-biphenyl-4-yl]-urea;
N-(4-Chloro-3-trifluoromethyl-phenyl)-N′-[4′-(N″,N″-dimethyl-amino-carbonyl)-3-(1H-tetrazol-5-yl)-biphenyl-4-yl]urea;
N-(4-Methoxyphenyl)-N′-[4′-(N″,N″-dimethyl-amino-carbonyl)-3-(1H-tetrazol-5-yl)-biphenyl-4-yl]urea;

or a pharmaceutically acceptable salt thereof.

In a seventh preferred embodiment the diphenyl urea derivative for use according to the invention is represented by Formula IV

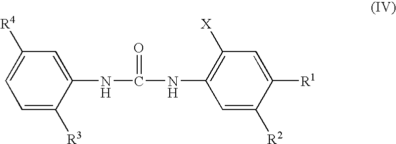

or a pharmaceutically acceptable salt thereof, wherein X, R¹ and R² are as defined above; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy, phenyl; or phenyl substituted with haloalkyl.

In a preferred embodiment X represents hydroxy or carboxy; R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and R² represents hydrogen, halo, haloalkyl, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy, phenyl; or phenyl substituted with haloalkyl.

In a more preferred embodiment X represents hydroxy or carboxy; R¹ represents hydrogen, halo, nitro, amino, N-phenyl-amino, N-benzoyl-amino, alkyl-carbonyl-amino or N-benzoyl-amino; R² represents hydrogen, halo, haloalkyl or nitro; R³ represents hydrogen, alkyl, halo, haloalkyl, nitro, hydroxy or alkoxy; and R⁴ represents hydrogen, halo, haloalkyl or nitro.

In a most preferred embodiment the diphenyl urea derivative for use according to the invention is
1,3-Bis-(2-hydroxy-5-trifluoromethyl-phenyl)-urea;

or a pharmaceutically acceptable salt thereof.

In an eight preferred embodiment the diphenyl urea derivative for use according to the invention is represented by Formula V

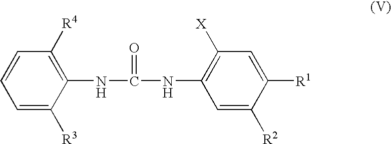

or a pharmaceutically acceptable salt thereof, wherein X, R¹ and R² are as defined above; R³ represents hydrogen, alkyl, halo, haloalkyl, nitro, hydroxy, alkoxy, phenyl or pyridyl; and R⁴ represents hydrogen, alkyl, halo, haloalkyl, nitro, hydroxy, alkoxy, phenyl or pyridyl.

In a preferred embodiment X represents a tetrazolyl group, an oxadiazolyl group or a triazolyl group; R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and R² represents hydrogen, halo, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy, phenyl; or phenyl substituted with haloalkyl.

In a ninth preferred embodiment the diphenyl urea derivative for use according to the invention is represented by Formula VI

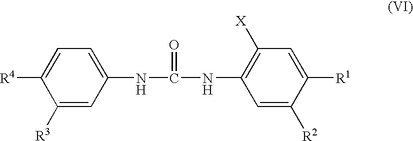

or a pharmaceutically acceptable salt thereof, wherein X, R¹ and R² are as defined above; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or R³ represents phenyl substituted with alkyl, halo or haloalkyl; and R⁴ represents hydrogen, alkyl, halo, haloalkyl, nitro, alkoxy, phenyl; or phenyl substituted with haloalkyl; or R³ and R⁴ together with the phenyl to which they are attached form a naphthyl group.

In a preferred embodiment X represents a tetrazolyl group, an oxadiazolyl group or a triazolyl group.

In a more preferred embodiment R¹ represents hydrogen, halo, hydroxy, alkoxy, nitro, amino, N-phenyl-amino, N-benzoyl-amino, alkyl-carbonyl-amino, N-benzoyl-amino, N,N-dialkyl acryl-amide, alkoxy-carbonyl, phenyl, naphthyl, pyridyl, furyl or thienyl; or R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, N,N-dialkyl-amino-carbonyl-alkyl, N-alkyl-N-acetic acid amino-carbonyl, anilino-carbonyl, piperidinyl-carbonyl, amino-carbonyl-N-alkyl-piperazine, N,N-dialkyl-sulfamoyl or sulfonamido-N-alkyl-piperazinium chloride; R² represents hydrogen, halo, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy or phenyl; or phenyl substituted with haloalkyl; or R³ and R⁴ together with the phenyl to which they are attached form a naphthyl group.

In an even more preferred embodiment X represents a tetrazolyl group, an oxadiazolyl group or a triazolyl group; R¹ represents halo, N,N-dialkyl acryl-amide, phenyl, phenyl substituted with halo, haloalkyl, N,N-dialkyl-amino-carbonyl-alkyl, piperidin-1-yl-carbonyl or N-alkyl-N-acetic acid amino-carbonyl; R² represents hydrogen; R³ represents halo, haloalkyl or nitro; and R⁴ represents alkyl or halo; or R³ and R⁴ together with the phenyl to which they are attached form a naphthyl group.

In a yet more preferred embodiment X represents tetrazolyl; R¹ represents halo, N,N-dialkyl acryl-amide, 2-halophenyl, 3-haloalkyl-phenyl, 4-haloalkyl-phenyl, 4-(2-N,N-dialkyl-amino-carbonyl-ethyl)-phenyl, 4-piperidin-1-yl-carbonyl-phenyl or N-methyl-N-acetic acid amino-carbonyl-phenyl; R² represents hydrogen; R³ represents halo, haloalkyl or nitro; and R⁴ represents halo or alkyl; or R³ and R⁴ together with the phenyl to which they are attached form a naphthyl group.

In a still more preferred embodiment X represents 1H-tetrazol-5-yl; R¹ represents bromo, N,N-dimethyl acryl-amide, 4-chlorophenyl, 4-fluorophenyl, 3-trifluoromethyl-phenyl, 4-trifluoromethyl-phenyl, 4-(2-N,N-dimethylcarbamoyl-ethyl)-phenyl, 4-piperidin-1-yl-carbonyl-phenyl or N-methyl-N-acetic acid amino-carbonyl-phenyl; R² represents hydrogen; R³ represents chloro, trifluoromethyl, or nitro; and R⁴ represents chloro, fluoro or methyl; or R³ and R⁴ together with the phenyl to which they are attached form a naphthyl group.

In a most preferred embodiment the diphenyl urea derivative for use according to the invention is
N-(3,4-Dichlorophenyl)-N′-[4-bromo-2-(1H-tetrazol-5-yl)phenyl]urea;
N-(4-Methyl-3-nitrophenyl)-N′-[4-bromo-2-(1H-tetrazol-5-yl)phenyl]urea;
N-(4-Chloro-3-trifluoromethylphenyl)-N′-[4-bromo-2-(1H-tetrazol-5-yl)phenyl]urea;
N-(4-Chloro-3-trifluoromethyl-phenyl)-N′-[3-(1H-tetrazol-5-yl)-4′-trifluoromethyl-biphenyl-4-yl]-urea;
N-(4-Chloro-3-trifluoromethyl-phenyl)-N′-[4′-chloro-3-(1H-tetrazol-5-yl)-biphenyl-4-yl]-urea;
N-(4-Chloro-3-trifluoromethyl-phenyl)-N′-[4′-fluoro-3-(1H-tetrazol-5-yl)-biphenyl-4-yl]-urea;
N-(4-Fluoro-3-trifluoromethyl-phenyl)-N′-[3-(1H-tetrazol-5-yl)-4′-trifluoromethyl-biphenyl-4-yl]-urea;
N-(4-Fluoro-3-trifluoromethyl-phenyl)-N′-[4′-chloro-3-(1H-tetrazol-5-yl)-biphenyl-4-yl]-urea;
N-(4-Fluoro-3-trifluoromethyl-phenyl)-N′-[4′-fluoro-3-(1H-tetrazol-5-yl)-biphenyl-4-yl]-urea;
N-(4-Fluoro-3-trifluoromethyl-phenyl)-N′-[3-(1H-tetrazol-5-yl)-3′-trifluoromethyl-biphenyl-4-yl]-urea;
N-(4-Fluoro-3-chloro-phenyl)-N′-(4′-(N,N-dimethylsulfamoyl)-2-(1H-tetrazol-5-yl)-4-biphenyl)urea;
N-(4-Chloro-3-trifluoromethyl-phenyl)-N′-(4′-(N,N-dimethylsulfamoyl)-2-(1H-tetrazol-5-yl)-4-biphenyl)urea;
N-(4-Fluoro-3-trifluoromethyl-phenyl)-N′-(4′-(N,N-dimethylsulfamoyl)-2-(1H-tetrazol-5-yl)-4-biphenyl)urea;
N-(4-Chloro-3-trifluoromethyl-phenyl)-N′-[4-(N″,N″-dimethyl acryl-amide)-2-(1-H-tetrazol-5-yl)-phenyl]urea;
N-(4-Chloro-3-trifluoromethyl-phenyl)-N′-[4′-(piperidine-1-carbonyl)-3-(1H-tetrazol-5-yl)-biphenyl-4-yl]urea;
N-(4-Chloro-3-trifluoromethyl-phenyl)-N′-{4′-[carbonyl-(N″-methyl)-amino-acetic acid]-2-(1H-tetrazol-5-yl)-4-biphenyl}urea;
N-(4-Trifluoromethyl-3-chloro-phenyl)-N′-[2-(1H-tetrazol-5-yl)-4-(2-N,N-dimethylcarbamoyl-ethyl)-phenyl]urea;
N-(4-Fluoro-3-trifluoromethyl-phenyl)-N′-[4-fluoro-2-(1H-tetrazol-5-yl)-phenyl]-urea; or
N-(2-Naphthyl)-N′-[4-bromo-2-(1H-tetrazol-5-yl)phenyl]urea;

or a pharmaceutically acceptable salt thereof.

In a tenth preferred embodiment the diphenyl urea derivative for use according to the invention is represented by Formula VII

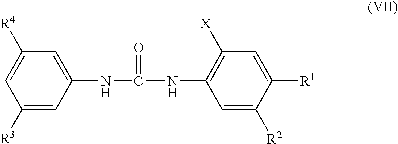

or a pharmaceutically acceptable salt thereof, wherein X, R¹ and R² are as defined above; R³ represents hydrogen, alkyl, halo, haloalkyl, haloalkoxy, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy or phenyl; or phenyl substituted with haloalkyl.

In a preferred embodiment X represents hydroxy or carboxy.

In a more preferred embodiment R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and R² represents hydrogen, halo, alkoxy, alkoxy-carbonyl, nitro, halophenyl, haloalkyl-phenyl or haloalkoxy-phenyl; R³ represents hydrogen, alkyl, halo, haloalkyl, haloalkoxy, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and R⁴ represents hydrogen, alkyl, halo, haloalkyl, nitro, alkoxy or phenyl; or phenyl substituted with haloalkyl.

In an even more preferred embodiment X represents hydroxy or carboxy; R¹ represents hydrogen, halo, hydroxy, alkoxy, nitro, alkoxy-carbonyl or N-phenyl-amino; R² represents hydrogen, halo, alkoxy, alkoxy-carbonyl or nitro; R³ represents alkyl, haloalkyl, haloalkoxy, nitro, hydroxy, carboxy, alkoxy-carbonyl, amino-carbonyl or benzoyl; and R⁴ represents hydrogen.

In a yet more preferred embodiment X represents hydroxy; R¹ represents hydrogen, halo, hydroxy, alkoxy, nitro, alkoxy-carbonyl or N-phenyl-amino; R² represents hydrogen, halo, alkoxy, alkoxy-carbonyl or nitro; R³ represents alkyl, haloalkyl, haloalkoxy, nitro, hydroxy, carboxy, alkoxy-carbonyl, amino-carbonyl or benzoyl; and R⁴ represents hydrogen.

In a still more preferred embodiment X represents hydroxy; R¹ represents hydrogen, chloro, hydroxy, methoxy, nitro, methoxy-carbonyl or N-phenyl-amino; R² represents hydrogen, chloro, methoxy, methoxy-carbonyl or nitro; R³ represents methyl, trifluoromethyl, trifluoromethoxy, nitro, hydroxy, carboxy, methoxy-carbonyl, amino-carbonyl or benzoyl; and R⁴ represents hydrogen.

In a most preferred embodiment the diphenyl urea derivative for use according to the invention is
N-(3-(Trifluoromethyl)phenyl)-N′-(2-hydroxyphenyl)urea;
N-(3-(Trifluoromethyl)phenyl)-N′-(2-hydroxy-4-methoxyphenyl)urea;
N-(3-(Trifluoromethyl)phenyl)-N′-(2-hydroxy-4-methoxycarbonylphenyl)urea;
N-(3-(Trifluoromethyl)phenyl)-N′-(2-hydroxy-4-chlorophenyl)urea;
N-(3-(Trifluoromethyl)phenyl)-N′-(2-hydroxy-4-nitrophenyl)urea;
N-(3-(Trifluoromethyl)phenyl)-N′-(2-hydroxy-4-(phenylamino)phenyl)urea;
N-(3-(Trifluoromethyl)phenyl)-N′-(2,4-dihydroxyphenyl)urea;
N-(3-(Trifluoromethyl)phenyl)-N′-(2-hydroxy-4-methoxycarbonyl-5-chlorophenyl)urea;
N-(3-(Trifluoromethoxy)phenyl)-N′-(2-hydroxy-5-chlorophenyl)urea;
N-(3-(Trifluoromethyl)phenyl)-N′-(2-hydroxy-5-methoxycarbonylphenyl)urea;
N-(3-(Trifluoromethyl)phenyl)-N′-(2-hydroxy-5-nitrophenyl)urea;
N-(3-(Trifluoromethyl)phenyl)-N′-(2-hydroxy-5-chlorophenyl)urea;
N-(3-(Trifluoromethyl)phenyl)-N′-(2-hydroxy-5-methoxyphenyl)urea;
N-(3-(Trifluoromethyl)phenyl)-N′-(2-hydroxy-5-nitrophenyl)urea;
N-(3-Benzoylphenyl)-N′-(2-hydroxy-5-chlorophenyl)urea;
N-(3-Carbamoylphenyl)-N′-(2-hydroxy-5-chlorophenyl)urea;
N-(3-Carboxyphenyl)-N′-(2-hydroxy-5-chlorophenyl)urea;
N-(3-Hydroxyphenyl)-N′-(2-hydroxy-5-chlorophenyl)urea;
N-(3-Methoxycarbonylphenyl)-N′-(2-hydroxy-5-chlorophenyl)urea;
N-(3-Methylphenyl)-N′-(2-hydroxy-5-chlorophenyl)urea; or
N-(3-Nitrophenyl)-N′-(2-hydroxy-5-chlorophenyl)urea;

or a pharmaceutically acceptable salt thereof.

In an eleventh preferred embodiment the diphenyl urea derivative for use according to the invention is represented by Formula VII, wherein X represents carboxy; R¹ represents halo or phenyl; R² represents hydrogen; R³ represents haloalkyl; and R⁴ represents hydrogen or haloalkyl.

In a preferred embodiment X represents carboxy; R¹ represents chloro, fluoro, bromo or phenyl; R² represents hydrogen; R³ represents trifluoromethyl; and R⁴ represents hydrogen or trifluoromethyl.

In a most preferred embodiment the diphenyl urea derivative for use according to the invention is
N-(3-Trifluoromethylphenyl)-N′-(2-carboxy-4-bromophenyl)urea;
N-(3-Trifluoromethylphenyl)-N′-(2-carboxy-4-chlorophenyl)urea;
N-(3-Trifluoromethylphenyl)-N′-(2-carboxy-4-fluorophenyl)urea;
N-(3-Trifluoromethylphenyl)-N′-(2-carboxy-4-trifluoromethylphenyl)urea;
N-(3-Trifluoromethylphenyl)-N′-(2-carboxy-4-biphenyl)urea; or
N-(3,5-Bis-trifluoromethylphenyl)-N′-(2-carboxy-4-biphenyl)urea;

or a pharmaceutically acceptable salt thereof.

In a twelfth preferred embodiment the diphenyl urea derivative for use according to the invention is represented by Formula VII, wherein X represents a tetrazolyl group; an oxadiazolyl group or a triazolyl group.

In a preferred embodiment R¹ represents hydrogen, halo, hydroxy, alkoxy, nitro, amino, N-phenyl-amino, alkyl-carbonyl-amino, N-benzoyl-amino, N,N-dialkyl acryl-amide, 2-N,N-dialkyl-carbamoyl-ethyl, alkyl-carbonyl, alkoxy-carbonyl, phenyl, naphthyl, pyridyl, furyl or thienyl; or R¹ represents phenyl substituted with halo, haloalkyl, haloalkoxy, nitro, carboxy, alkoxy-carbonyl, amino-carbonyl (carbamoyl), N,N-dialkyl-amino-carbonyl, N-phenyl-amino-carbonyl, anilino-carbonyl, N-acetic acid-amino-carbonyl, N-alkyl-N-acetic acid-amino-carbonyl, carbamoyl-N-alkyl-piperazine, N,N-dialkyl-sulfamoyl or sulfonamido-N-alkyl-piperazinium chloride; and R² represents hydrogen, halo, alkoxy, alkoxy-carbonyl, nitro, halophenyl, haloalkyl-phenyl or haloalkoxy-phenyl; R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl or pyridyl; or R³ represents phenyl substituted with alkyl, halo or haloalkyl; and R⁴ represents hydrogen, alkyl, halo, haloalkyl, nitro, alkoxy, phenyl or phenyl substituted with haloalkyl.

In a more preferred embodiment X represents tetrazolyl; R¹ represents hydrogen, halo, nitro, amino, alkyl-carbonyl, alkyl-carbonyl-amino, N-benzoyl-amino, phenyl, naphthyl, pyridyl, furyl or thienyl; or R¹ represents phenyl substituted with halo, haloalkyl, nitro, carboxy, alkoxy-carbonyl, amino-carbonyl, N,N-dialkyl-amino-carbonyl, N-phenyl-amino-carbonyl, N,N-dialkyl-sulfamoyl, N-phenyl-amino-carbonyl, sulfonamido-N-alkyl-piperazinium chloride, carbamoyl-N-alkyl-piperazine, anilino-carbonyl; and R² represents hydrogen; R³ represents alkyl, halo, haloalkyl, nitro, alkoxy, alkyl-carbonyl, phenyl or pyridyl; and R⁴ represents hydrogen.

In an even more preferred embodiment X represents tetrazol; R¹ represents hydrogen, halo, nitro, amino, alkyl-carbonyl-amino, N-benzoyl-amino, phenyl, naphthyl, pyridyl, furyl or thienyl; or R¹ represents phenyl substituted at position 3 or 4 with halo, haloalkyl, nitro, alkoxy-carbonyl, amino-carbonyl, N,N-dialkyl-amino-carbonyl, N-phenyl-amino-carbonyl, carboxy, benzoylamino, anilino-carbonyl, N,N-dialkyl-sulfamoyl, carbamoyl-N-alkyl-piperazine, sulfonamido-N-alkyl-piperazinium chloride; and R² represents hydrogen; R³ represents halo, haloalkyl, acetyl, phenyl or pyridyl; and R⁴ represents hydrogen.

In a yet more preferred embodiment X represents tetrazol; R¹ represents hydrogen, halo, nitro, amino, alkyl-carbonyl-amino, N-benzoyl-amino, phenyl, naphthyl, pyridyl, furyl or thienyl; or R¹ represents phenyl substituted at position 3 with nitro, or at position 4 with halo, haloalkyl, alkoxy-carbonyl, amino-carbonyl, N,N-dialkyl-amino-carbonyl, N-phenyl-amino-carbonyl, benzoylamino, carboxy, anilino-carbonyl, N,N-dialkyl-sulfamoyl, carbamoyl-N-alkyl-piperazine or sulfonamido-N-alkyl-piperazinium chloride; and R² represents hydrogen; R³ represents alkyl, halo, haloalkyl, alkyl-carbonyl, phenyl or pyridyl; and R⁴ represents hydrogen.

In a still more preferred embodiment X represents 1H-tetrazol-5-yl; R¹ represents hydrogen, fluoro, bromo, nitro, amino, acetyl-amino, N-benzoyl-amino, phenyl, 3-nitrophenyl, 4-chlorophenyl, 4-fluorophenyl, 4-trifluoromethyl-phenyl, 4-(ethoxy-carbonyl)-phenyl, 4-carboxy-phenyl, 4-benzoylamino-phenyl, 4-(amino-carbonyl)-phenyl, 4-(N,N-dimethyl-amino-carbonyl)-phenyl, 4-(N,N-diethyl-amino-carbonyl)-phenyl, 4-(N-phenyl-amino-carbonyl)-phenyl, 4-(anilino-carbonyl)-phenyl, 4-(N,N-dimethyl-sulfamoyl)-phenyl, 4-(sulfonamido-N-methyl-piperazinium chloride)-phenyl or 4-(carbamoyl-N-methyl-piperazine)-phenyl, 1-naphthyl, 2-naphthyl, 3-pyridyl, 3-furyl or 3-thienyl; R² represents hydrogen; R³ represents methyl, chloro, fluoro, bromo, trifluoromethyl, nitro, methoxy, acetyl, phenyl or 3-pyridyl; and R⁴ represents hydrogen.

In a most preferred embodiment the diphenyl urea derivative for use according to the invention is
N-3-Trifluoromethylphenyl-N′-2-(1H-tetrazol-5-yl)phenyl urea;
N-3-Trifluoromethylphenyl-N′-4-nitro-2-(1H-tetrazol-5-yl)phenyl urea;
N-3-Trifluoromethylphenyl-N′-4-(1-naphthyl)-2-(1H-tetrazol-5-yl)phenyl urea;
N-3-Trifluoromethylphenyl-N′-4-(2-naphthyl)-2-(1H-tetrazol-5-yl)phenyl urea;
N-3-Trifluoromethylphenyl-N′-4-(3-pyridyl)-2-(1H-tetrazol-5-yl)phenyl urea;
N-3-Trifluoromethylphenyl-N′-4-(4-trifluoromethylphenyl)-2-(1H-tetrazol-5-yl)phenyl urea;
N-3-Trifluoromethylphenyl-N′-4-(3-furyl)-2-(1H-tetrazol-5-yl)phenyl urea;
N-3-Trifluoromethylphenyl-N′-4-(3-thienyl)-2-(1H-tetrazol-5-yl)phenyl urea;
N-3-Trifluoromethylphenyl-N′-4-(3-nitrophenyl)-2-(1H-tetrazol-5-yl)phenyl urea;
N-3-Trifluoromethylphenyl-N′-4-(4-ethoxycarbonylphenyl)-2-(1H-tetrazol-5-yl)phenyl urea;
N-3-Trifluoromethylphenyl-N′-4-(4-aminocarbonylphenyl)-2-(1H-tetrazol-5-yl)phenyl urea;
N-3-Trifluoromethylphenyl-N′-4-(4-diethylaminocarbonylphenyl)-2-(1H-tetrazol-5-yl)phenyl urea;
N-3-Trifluoromethylphenyl-N′-4-(4-phenylaminocarbonylphenyl)-2-(1H-tetrazol-5-yl)phenyl urea;
N-3-Trifluoromethylphenyl-N′-4-(4-benzoylamino-phenyl)-2-(1H-tetrazol-5-yl)phenyl urea;
N-3-Trifluoromethylphenyl-N′-4-amino-2-(1H-tetrazol-5-yl)phenyl urea;
N-3-Trifluoromethylphenyl-N′-4-acetylamino-2-(1H-tetrazol-5-yl)phenyl urea;
N-3-Trifluoromethylphenyl-N′-4-benzoylamino-2-(1H-tetrazol-5-yl)phenyl urea;
N-3-Trifluoromethylphenyl-N′-4-(4-carboxyphenyl)-2-(1H-tetrazol-5-yl)phenyl urea;
N-3-Trifluoromethylphenyl-N′-4-(4-anilinocarbonylphenyl)-2-(1H-tetrazol-5-yl)phenyl urea;
N-3-Biphenylyl-N′-2-(1H-tetrazol-5-yl)phenyl urea;
N-3-Bromophenyl-N′-4-bromo-2-(1H-tetrazol-5-yl)phenyl urea;
N-3-Acetylphenyl-N′-2-(1H-tetrazol-5-yl)phenyl urea;
N-3-Biphenylyl-N′-4-bromo-2-(1H-tetrazol-5-yl)phenyl urea; or
N-3-(3-Pyridyl)phenyl-N′-4-bromo-2-(1H-tetrazol-5-yl)phenyl urea;
N-(3-Bromophenyl)-N′-(2-(1H-tetrazol-5-yl)-4-biphenyl)urea;
N-(3-Trifluoromethylphenyl)-N′-(2-(1H-tetrazol-5-yl)-4-biphenyl)urea;
N-(3-Trifluoromethylphenyl)-N′-(4′-(N,N-dimethylsulfamoyl)-2-(1H-tetrazol-5-yl)-4-biphenyl)urea;
N-(3-Bromophenyl)-N′-(4′-(N,N-dimethylsulfamoyl)-2-(1H-tetrazol-5-yl)-4-biphenyl)urea;
N-(3-Bromophenyl)-N′-(4′-(N,N-dimethylcarbamoyl)-2-(1H-tetrazol-5-yl)-4-biphenyl)urea;
N-(3-Trifluoromethylphenyl)-N′-(4-amino-2-(1H-tetrazol-5-yl)phenyl)urea;
N-(3-Trifluoromethylphenyl)-N′-(4-acetylamino-2-(1H-tetrazol-5-yl)phenyl)urea;
N-(3-Trifluoromethylphenyl)-N′-(4′-carbamoyl-2-(1H-tetrazol-5-yl)-4-biphenyl)urea;
N-(3-Trifluoromethylphenyl)-N′-(4′-(N,N-dimethylcarbamoyl)-2-(1H-tetrazol-5-yl)-4-biphenyl)urea;
N-(3-Trifluoromethylphenyl)-N′-(4′-carboxy-2-(1H-tetrazol-5-yl)-4-biphenyl)urea;
N-(3-Trifluoromethylphenyl)-N′-(4′-(N-phenylcarbamoyl)-2-(1H-tetrazol-5-yl)-4-biphenyl)urea;
N-(3-Biphenyl)-N′-(2-(1H-tetrazol-5-yl)phenyl)urea;
N-(3-Acetylphenyl)-N′-(2-(1H-tetrazol-5-yl)phenyl)urea;
N-(3-Biphenyl)-N′-(4-bromo-2-(1H-tetrazol-5-yl)phenyl)urea;
N-(3-(3-Pyridyl)phenyl)-N′-(4-bromo-2-(1H-tetrazol-5-yl)phenyl)urea;
N-(3-Bromophenyl)-N′-(4-bromo-2-(1H-tetrazol-5-yl)phenyl)urea;
N-(3-Trifluoromethylphenyl)-N′-4-(4-benzoylcarbonylphenyl)-2-(1H-tetrazol-5-yl)phenyl urea;
N-(3-Bromophenyl)-N′-[3′-nitro-2-(1H-tetrazol-5-yl)biphenyl]urea;
N-(3-Bromophenyl)-N′-[4′-(sulfonamido-N′-methylpiperazinium chloride)-2-(1H-tetrazol-5-yl)-4-biphenyl]urea;
N-(3-Bromophenyl)-N′-[4′-carbamoyl-N′-methylpiperazine)-2-(1H-tetrazol-5-yl)-4′-biphenyl]urea;
N-(3-Methoxyphenyl)-N′-[4-bromo-2-(1H-tetrazol-5-yl)phenyl]urea;
N-(3-Chlorophenyl)-N′-[4-bromo-2-(1H-tetrazol-5-yl)phenyl]urea;
N-(3-Methylphenyl)-N′-[4-bromo-2-(1H-tetrazol-5-yl)phenyl]urea;
N-(3-Fluorophenyl)-N′-[4-bromo-2-(1H-tetrazol-yl)phenyl]urea;
N-(3-Nitrophenyl)-N′-[4-bromo-2-(1H-tetrazol-5-yl)phenyl]urea;
N-(3-Acetylphenyl)-N′-[4-bromo-2-(1H-tetrazol-5-yl)phenyl]urea;
N-(3-Trifluoromethylphenyl)-N′-[4-fluoro-2-(1H-tetrazol-yl)phenyl]urea;
N-(3-Trifluoromethylphenyl)-N′-[4-bromo-2-(1H-tetrazol-yl)phenyl]urea;
N-(3-Trifluoromethylphenyl)-N′-[3-(1H-tetrazol-5-yl)-4′-trifluoromethyl-biphenyl-4-yl]-urea;
N-(3-Trifluoromethylphenyl)-N′-[4′-chloro-3-(1H-tetrazol-5-yl)-biphenyl-4-yl]-urea;
N-(3-Trifluoromethylphenyl)-N′-[4′-fluoro-3-(1H-tetrazol-5-yl)-biphenyl-5-yl]-urea;
N-(3-Bromophenyl)-N′-[3-(1H-tetrazol-5-yl)-4′-trifluoromethyl-biphenyl-4-yl]-urea;

or a pharmaceutically acceptable salt thereof.

In a thirteenth preferred embodiment the diphenyl urea derivative for use according to the invention is represented by Formula VII, wherein X represents tetrazolyl; R¹ represents halo, N,N-dialkyl acryl-amide, N,N-dialkyl-amino-carbonyl-alkyl or phenyl; or R¹ represents phenyl substituted in position 3 or 4 with halo, haloalkyl, haloalkoxy, amino-carbonyl, N,N-dialkyl-sulfamoyl, N,N-dialkyl-amino-carbonyl, N-acetic acid-amino-carbonyl, N-alkyl-N-acetic acid-amino-carbonyl or anilino-carbonyl; and R² represents hydrogen; R³ represents alkyl, halo or haloalkyl; and R⁴ represents alkyl, halo or haloalkyl.

In a preferred embodiment X represents tetrazolyl; R¹ represents halo, N,N-dialkyl acryl-amide, N,N-dialkyl-amino-carbonyl-alkyl, 4-halophenyl, 3-haloalkyl-phenyl, 4-haloalkyl-phenyl, 4-haloalkoxy-phenyl, 4-N,N-dialkyl-sulfamoyl-phenyl, 4-N,N-dialkyl-amino-carbonyl-phenyl, 4-amino-carbonyl-phenyl, N-acetic acid-amino-carbonyl-phenyl, N-alkyl-N-acetic acid-amino-carbonyl-phenyl or 4-anilino-carbonyl-phenyl; R² represents hydrogen; R³ represents alkyl, halo or haloalkyl; and R⁴ represents alkyl, halo or haloalkyl.

In a more preferred embodiment X represents 1H-tetrazol-5-yl; R¹ represents bromo, N,N-dimethyl acryl-amide, 2-N,N-dimethyl-carbamoyl-ethyl, 4-chlorophenyl, 4-fluorophenyl, 3-trifluoromethyl-phenyl, 4-trifluoromethyl-phenyl, 4-methoxy-phenyl, 4-N,N-dimethylsulfamoyl-phenyl, 4-N,N-dimethylcarbamoyl-phenyl, N-acetic acid-amino-carbonyl-phenyl, 4-amino-carbonyl-phenyl, N-methyl-N-acetic acid-amino-carbonyl-phenyl or 4-anilinocarbonyl-phenyl; R² represents hydrogen; R³ represents methyl, chloro, fluoro or trifluoromethyl; and R⁴ represents methyl, chloro, fluoro or trifluoromethyl.

In an even more preferred embodiment X represents 1H-tetrazol-5-yl; R¹ represents 3-trifluoromethyl-phenyl, 4-fluorophenyl, 4-trifluoromethyl-phenyl, 4-methoxy-phenyl, 4-trifluoromethoxy-phenyl, R² represents hydrogen; R³ represents chloro, fluoro or trifluoromethyl; and R⁴ represents chloro, fluoro or trifluoromethyl.

In a most preferred embodiment the diphenyl urea derivative for use according to the invention is
N-(3,5-Dichlorophenyl)-N′-[4-bromo-2-(1H-tetrazol-5-yl)phenyl]urea;
N-(3,5-Bis-trifluoromethylphenyl)-N′-[4-bromo-2-(1H-tetrazol-5-yl)phenyl]urea;
N-(3,5-Bis-trifluoromethylphenyl)-N′-[4-chloro-2-(1H-tetrazol-5-yl)phenyl]urea;
N-(3,5-Dichlorophenyl)-N′-[4-chloro-2-(1H-tetrazol-5-yl)phenyl]urea;
N-(3,5-Difluorophenyl)-N′-[4′-chloro-2-(1H-tetrazol-5-yl)phenyl]urea;
N-(3,5-Dimethylphenyl)-N′-[4-bromo-2-(1H-tetrazol-5-yl)phenyl]urea;
N-(3,5-Dichlorophenyl)-N′-[4′-chloro-3-(1H-tetrazol-5-yl)-biphenyl-4-yl]-urea;
N-(3,5-Difluorophenyl)-N′-[4′-chloro-3-(1H-tetrazol-5-yl)-biphenyl-4-yl]-urea;
N-(3,5-Dichlorophenyl)-N′-[3-(1H-tetrazol-5-yl)-4′-trifluoromethyl-biphenyl-4-yl]-urea;
N-(3,5-Difluorophenyl)-N′-[3-(1H-tetrazol-5-yl)-4′-trifluoromethyl-biphenyl-4-yl]-urea;
N-(3,5-Dichlorophenyl)-N′-[4′-fluoro-3-(1H-tetrazol-5-yl)-biphenyl-4-yl]-urea;
N-(3,5-Difluorophenyl)-N′-[4′-fluoro-2-(1H-tetrazol-5-yl)-biphenyl-4-yl]-urea;
N-(3,5-Bis-trifluoromethylphenyl)-N′-[3-(1H-tetrazol-5-yl)-4′-trifluoromethyl-biphenyl-4-yl]-urea;
N-(3,5-Bis-trifluoromethylphenyl)-N′-[4′-chloro-3-(1H-tetrazol-5-yl)-biphenyl-4-yl]-urea;
N-(3,5-Bis-trifluoromethylphenyl)-N′-[4′-fluoro-3-(1H-tetrazol-5-yl)-biphenyl-4-yl]-urea;
N-(3,5-Dichlorophenyl)-N′-[4′-methoxy-3-(1H-tetrazol-5-yl)-biphenyl-4-yl]-urea;
N-(3,5-Difluorophenyl)-N′-[3-(1H-tetrazol-5-yl)-4′-trifluoromethoxy-biphenyl-4-yl]-urea;
N-(3,5-Dichlorophenyl)-N′-[3-(1H-tetrazol-5-yl)-4′-trifluoromethoxy-biphenyl-4-yl]-urea;
N-(3,5-Bis-trifluoromethylphenyl)-N′-[3-(1H-tetrazol-5-yl)-4′-trifluoromethoxy-biphenyl-4-yl]-urea;
N-(3,5-Difluorophenyl)-N′-[3-(1H-tetrazol-5-yl)-3′-trifluoromethyl-biphenyl-4-yl]-urea;
N-(3,5-Dichlorophenyl)-N′-[3-(1H-tetrazol-5-yl)-3′-trifluoromethyl-biphenyl-4-yl]-urea;
N-(3,5-Bis-trifluoromethylphenyl)-N′-[3-(1H-tetrazol-5-yl)-3′-trifluoromethyl-biphenyl-4-yl]-urea;
N-(3,5-Difluorophenyl)-N′-[4′-methoxy-3-(1H-tetrazol-5-yl)-biphenyl-4-yl]-urea;
N-(3,5-Bis-trifluoromethylphenyl)-N′-[4′-methoxy-3-(1H-tetrazol-5-yl)-biphenyl-4-yl]-urea;
N-(3,5-Bis-trifluoromethylphenyl)-N′-(4′-(N,N-dimethylsulfamoyl)-2-(1H-tetrazol-5-yl)-4-biphenyl)urea;
N-(3,5-Difluorophenyl)-N′-(4′-carbamoyl-2-(1H-tetrazol-5-yl)-4-biphenyl)urea;
N-(3,5-Dichlorophenyl)-N′-(4′-(N,N-dimethylcarbamoyl)-2-(1H-tetrazol-5-yl)-4-biphenyl)urea;
N-(3,5-Bis-trifluoromethylphenyl)-N′-(4′-(N,N-dimethylcarbamoyl)-2-(1H-tetrazol-5-yl)-4-biphenyl)urea;
N-(3,5-Bis-trifluoromethylphenyl)-N′-(4′-carbamoyl-2-(1H-tetrazol-5-yl)-4-biphenyl)urea;
N-(3,5-Dichlorophenyl)-N′-(4′-(N,N-dimethylsulfamoyl)-2-(1H-tetrazol-5-yl)-4-biphenyl)urea;
N-(3,5-Difluorophenyl)-N′-(4′-(N,N-dimethylsulfamoyl)-2-(1H-tetrazol-5-yl)-4-biphenyl)urea;
N-(3-Trifluoromethyl-5-fluoro-phenyl)-N′-(4′-(N,N-dimethylsulfamoyl)-2-(1H-tetrazol-5-yl)-4-biphenyl)urea;
N-(3,5-Bis-trifluoromethylphenyl)-N′-4-(4-anilinocarbonylphenyl)-2-(1H-tetrazol-5-yl)phenyl urea;
N-(3,5-Bis-trifluoromethyl-phenyl)-N′-{4′-[carbonyl-amino-acetic acid]-2-(1H-tetrazol-5-yl)-4-biphenyl}urea;
N-(3,5-Difluorophenyl)-N′-{4′-[carbonyl-(N″-methyl)-amino-acetic acid]-2-(1H-tetrazol-5-yl)-4-biphenyl}urea;
N-(3,5-Bis-trifluoromethyl-phenyl)-N′-{4′-[carbonyl-(N″-methyl)-amino-acetic acid]-2-(1H-tetrazol-5-yl)-4-biphenyl}urea;
N-(3,5-Dichloro-phenyl)-N′-[4-(N″,N″-dimethyl acryl-amide)-2-(1-H-tetrazol-5-yl)-phenyl]urea;
N-(3,5-Dichloro-phenyl)-N′-[2-(1H-tetrazol-5-yl)-4-(2-N,N-dimethyl-carbamoyl-ethyl)-phenyl]urea;
N-(3,5-Bis-trifluoromethylphenyl)-N′-[2-(1H-tetrazol-5-yl)-4-(2-N,N-dimethyl-carbamoyl-ethyl)-phenyl]urea;

or a pharmaceutically acceptable salt thereof.

In a fourteenth preferred embodiment the diphenyl urea derivative for use according to the invention is represented by Formula VII, wherein X represents an oxadiazolyl group; R¹ represents hydrogen; R² represents hydrogen; R³ represents haloalkyl; and R⁴ represents hydrogen.

In a preferred embodiment X represents 2-oxo-3H-1,3,4-oxadiazol-5-yl or 5-oxo-4,5-dihydro-[1,2,4]oxadiazol-3-yl; R¹ represents hydrogen; R² represents hydrogen; R³ represents trifluoromethyl; and R⁴ represents hydrogen.

In a most preferred embodiment the diphenyl urea derivative for use according to the invention is
N-(3-Trifluoromethylphenyl)-N′-2-(2-oxo-3H-1,3,4-oxadiazol-5-yl)phenyl urea; or
N-(3-Trifluoromethylphenyl)-N′-[2-(5-oxo-4,5-dihydro-[1,2,4]oxadiazol-3-yl)-4-(4′-N,N-dimethyl-carbamoyl)-biphenyl]urea;

or a pharmaceutically acceptable salt thereof.

In a fifteenth preferred embodiment the diphenyl urea derivative for use according to the invention is represented by Formula VII, wherein X represents 4-hydroxy-1,2,4-triazol-3-yl or 3-oxo-1,2-dihydro-1,2,4-triazol-1-yl; R¹ represents hydrogen or phenyl; R² represents hydrogen; R³ represents trifluoromethyl; and R⁴ represents hydrogen.

In a most preferred embodiment the diphenyl urea derivative for use according to the invention is
N-3-Trifluoromethylphenyl-N′-2-(4-hydroxy-1,2,4-triazol-3-yl)phenyl urea;
N-3-Trifluoromethylphenyl-N′-2-(3-oxo-1,2-dihydro-1,2,4-triazol-1-yl)phenyl urea; or
N-3-Trifluoromethylphenyl-N′-4-biphenylyl-2-(3-oxo-1,2-dihydro-1,2,4-triazol-1-yl)phenyl urea;

or a pharmaceutically acceptable salt thereof.

In a sixteenth preferred embodiment the diphenyl urea derivative for use according to the invention is represented by Formula VIII,

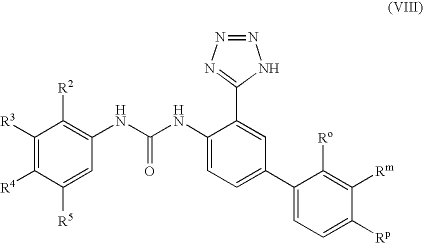

or a pharmaceutically acceptable salt thereof, wherein R^{o}, R^{m} and R^{P} independently of each other represent hydrogen, halo, trifluoromethyl, trifluoromethoxy, alkyl or alkoxy; with the proviso that not all three of R^{o}, R^{m} and R^{P} represent hydrogen; R², R³, R⁴ and R⁵ independently of each other represent hydrogen, halo, trifluoromethyl, trifluoromethoxy, alkyl or alkoxy; with the proviso that the compound is not N-(3-Trifluoromethyl-phenyl)-N′-[3-(1H-tetrazol-5-yl)-4′-trifluoromethyl-biphenyl-4-yl]-urea.

In a preferred embodiment R^{o} represents hydrogen; R^{m} represents hydrogen; and R^{P} represents halo, trifluoromethyl, trifluoromethoxy, alkyl or alkoxy. In a special embodiment, R^{P} represents halo, such as chloro or fluoro, or bromo. In a further embodiment, R^{P} represents trifluoromethyl. In a still further embodiment, R^{P} represents trifluoromethoxy. In a further embodiment, R^{P} represents alkyl, such as methyl. In a still further embodiment, R^{P} represents alkoxy, such as methoxy.

In another preferred embodiment R^{o} represents hydrogen; R^{P} represents hydrogen; and R^{m} represents halo, trifluoromethyl, trifluoromethoxy, alkyl or alkoxy. In a special embodiment, R^{m} represents trifluoromethyl.

In an even more preferred embodiment R³, R⁴ and R⁵ represent hydrogen; and R² represents halo, trifluoromethyl, trifluoromethoxy, alkyl or alkoxy. In a special embodiment, R² represents halo, such as chloro, fluoro or bromo. In a further embodiment, R² represents trifluoromethyl.

In a further embodiment R², R⁴ and R⁵ represent hydrogen; and R³ represents halo, trifluoromethyl, trifluoromethoxy, alkyl or alkoxy. In a special embodiment, R³ represents trifluoromethyl. In a further embodiment, R³ represents halo, such as bromo.

In a still further embodiment R², R³ and R⁵ represent hydrogen; and R⁴ represents halo, trifluoromethyl, trifluoromethoxy, alkyl or alkoxy. In a special embodiment, R⁴ represents halo, such as chloro.

In a yet further embodiment two of R², R³, R⁴ and R⁵ represent hydrogen, and the other two of R², R³, R⁴ and R⁵ independently of each other represent halo, trifluoromethyl, trifluoromethoxy, alkyl or alkoxy.

In a still further embodiment of the compound of general formula I, R² and R⁵ represent hydrogen; and R³ and R⁴ independently of each other represent halo, trifluoromethyl, trifluoromethoxy, alkyl or alkoxy. In a special embodiment, R³ represents trifluoromethyl. In a further embodiment, R⁴ represents halo, such as chloro or fluoro. In a still further embodiment, R³ represents trifluoromethyl and R⁴ represents chloro. In a further embodiment, R³ represents trifluoromethyl and R⁴ represents fluoro.

In a still further embodiment of the compound of general formula I, R² and R⁴ represent hydrogen; and R³ and R⁵ independently of each other represent halo, trifluoromethyl, trifluoromethoxy, alkyl or alkoxy. In a special embodiment, R³ represents trifluoromethyl. In a further embodiment, R³ represents halo, such as chloro or fluoro. In a still further embodiment, R⁵ represents trifluoromethyl. In a further embodiment, R⁵ represents halo, such as chloro or fluoro. In a still further embodiment, R³ represents chloro and R⁵ represents chloro. In a further embodiment, R³ represents fluoro and R⁵ represents fluoro. In a still further embodiment, R³ represents trifluoromethyl and R⁵ represents trifluoromethyl.

In a most preferred embodiment the diphenyl urea derivative for use according to the invention is
N-(4-Chloro-3-trifluoromethyl-phenyl)-N′-[3-(1H-tetrazol-5-yl)-4′-trifluoromethyl-biphenyl-4-yl]-urea;
N-(3-Trifluoromethyl-phenyl)-N′-[4′-chloro-3-(1H-tetrazol-5-yl)-biphenyl-4-yl]-urea;
N-(4-Chloro-3-trifluoromethyl-phenyl)-N′-[4′-chloro-3-(1H-tetrazol-5-yl)-biphenyl-4-yl]-urea;
N-(3,5-Dichloro-phenyl)-N′-[4′-chloro-3-(1H-tetrazol-5-yl)-biphenyl-4-yl]-urea;
N-(3,5-Difluoro-phenyl)-N′-[4′-chloro-3-(1H-tetrazol-5-yl)-biphenyl-4-yl]-urea;
N-(3,5-Dichloro-phenyl)-N′-[3-(1H-tetrazol-5-yl)-4′-trifluoromethyl-biphenyl-4-yl]-urea;
N-(3,5-Difluoro-phenyl)-N′-[3-(1H-tetrazol-5-yl)-4′-trifluoromethyl-biphenyl-4-yl]-urea;
N-(3-Trifluoromethyl-phenyl)-N′-[4′-fluoro-3-(1H-tetrazol-5-yl)-biphenyl-5-yl]-urea;
N-(4-Chloro-3-trifluoromethyl-phenyl)-N′-[4′-fluoro-3-(1H-tetrazol-5-yl)-biphenyl-4-yl]-urea;
N-(3,5-Dichloro-phenyl)-N′-[4′-fluoro-3-(1H-tetrazol-5-yl)-biphenyl-4-yl]-urea;
N-(3,5-Difluoro-phenyl)-N′-[4′-fluoro-2-(1H-tetrazol-5-yl)-biphenyl-4-yl]-urea;
N-(4-Fluoro-3-trifluoromethyl-phenyl)-N′-[3-(1H-tetrazol-5-yl)-4′-trifluoromethyl-biphenyl-4-yl]-urea;
N-(3,5-Bis-trifluoromethyl-phenyl)-N′-[3-(1H-tetrazol-5-yl)-4′-trifluoromethyl-biphenyl-4-yl]-urea;
N-(3,5-Bis-trifluoromethyl-phenyl)-N′-[4′-chloro-3-(1H-tetrazol-5-yl)-biphenyl-4-yl]-urea;
N-(4-Fluoro-3-trifluoromethyl-phenyl)-N′-[4′-chloro-3-(1H-tetrazol-5-yl)-biphenyl-4-yl]-urea;
N-(3,5-Bis-trifluoromethyl-phenyl)-N′-[4′-fluoro-3-(1H-tetrazol-5-yl)-biphenyl-4-yl]-urea;
N-(4-Fluoro-3-trifluoromethyl-phenyl)-N′-[4′-fluoro-3-(1H-tetrazol-5-yl)-biphenyl-4-yl]-urea;
N-(2-Trifluoromethyl-phenyl)-N′-[3-(1H-tetrazol-5-yl)-4′-trifluoromethyl-biphenyl-4-yl]-urea;
N-(2-Trifluoromethyl-phenyl)-N′-[4′-fluoro-3-(1H-tetrazol-5-yl)-biphenyl-4-yl]-urea;
N-(2-Bromo-phenyl)-N′-[3-(1H-tetrazol-5-yl)-4′-trifluoromethyl-biphenyl-4-yl]-urea;
N-(2-Trifluoromethyl-phenyl)-N′-[4′-chloro-3-(1H-tetrazol-5-yl)-biphenyl-4-yl]-urea;
N-(2-Bromo-phenyl)-N′-[4′-chloro-3-(1H-tetrazol-5-yl)-biphenyl-4-yl]-urea;
N-(2-Bromo-phenyl)-N′-[4′-fluoro-3-(1H-tetrazol-5-yl)-biphenyl-4-yl]-urea;
N-(2-Fluoro-phenyl)-N′-[3-(1H-tetrazol-5-yl)-4′-trifluoromethyl-biphenyl-4-yl]-urea;
N-(2-Fluoro-phenyl)-N′-[4′-chloro-3-(1H-tetrazol-5-yl)-biphenyl-4-yl]-urea;
N-(2-Fluoro-phenyl)-N′-[4′-fluoro-3-(1H-tetrazol-5-yl)-biphenyl-4-yl]-urea;
N-(2-Fluoro-phenyl)-N′-[4′-methyl-3-(1H-tetrazol-5-yl)-biphenyl-4-yl]-urea;
N-(2-Chloro-phenyl)-N′-[4′-methyl-3-(1H-tetrazol-5-yl)-biphenyl-4-yl]-urea;
N-(2-Bromo-phenyl)-N′-[4′-methyl-3-(1H-tetrazol-5-yl)-biphenyl-4-yl]-urea;
N-(2-Trifluoromethyl-phenyl)-N′-[4′-methyl-3-(1H-tetrazol-5-yl)-biphenyl-4-yl]-urea;
N-(2-Chloro-phenyl)-N′-[3-(1H-tetrazol-5-yl)-4′-trifluoromethyl-biphenyl-4-yl]-urea;
N-(2-Chloro-phenyl)-N′-[4′-chloro-3-(1H-tetrazol-5-yl)-biphenyl-4-yl]-urea;
N-(2-Chloro-phenyl)-N′-[4′-fluoro-3-(1H-tetrazol-5-yl)-biphenyl-4-yl]-urea;
N-(3,5-Dichloro-phenyl)-N′-[4′-methoxy-3-(1H-tetrazol-5-yl)-biphenyl-4-yl]-urea;
N-(3,5-Difluoro-phenyl)-N′-[3-(1H-tetrazol-5-yl)-4′-trifluoromethoxy-biphenyl-4-yl]-urea;
N-(3,5-Dichloro-phenyl)-N′-[3-(1H-tetrazol-5-yl)-4′-trifluoromethoxy-biphenyl-4-yl]-urea;
N-(3,5-Bis-trifluoromethyl-phenyl)-N′-[3-(1H-tetrazol-5-yl)-4′-trifluoromethoxy-biphenyl-4-yl]-urea;
N-(3,5-Difluoro-phenyl)-N′-[3-(1H-tetrazol-5-yl)-3′-trifluoromethyl-biphenyl-4-yl]-urea;
N-(3,5-Dichloro-phenyl)-N′-[3-(1H-tetrazol-5-yl)-3′-trifluoromethyl-biphenyl-4-yl]-urea;
N-(3,5-Bis-trifluoromethyl-phenyl)-N′-[3-(1H-tetrazol-5-yl)-3′-trifluoromethyl-biphenyl-4-yl]-urea;
N-(3,5-Difluoro-phenyl)-N′-[4′-methoxy-3-(1H-tetrazol-5-yl)-biphenyl-4-yl]-urea;
N-(3,5-Bis-trifluoromethyl-phenyl)-N′-[4′-methoxy-3-(1H-tetrazol-5-yl)-biphenyl-4-yl]-urea;
N-(3-Bromo-phenyl)-N′-[3-(1H-tetrazol-5-yl)-4′-trifluoromethyl-biphenyl-4-yl]-urea;
N-(4-Chloro-phenyl)-N′-[3-(1H-tetrazol-5-yl)-4′-trifluoromethyl-biphenyl-4-yl]-urea;
N-(4-Fluoro-3-trifluoromethyl-phenyl)-N′-[3-(1H-tetrazol-5-yl)-3′-trifluoromethyl-biphenyl-4-yl]-urea;

or a pharmaceutically acceptable salt thereof.

Definition of Substituents

In the context of this invention halo represents fluoro, chloro, bromo or iodo, and haloalkyl, haloalkoxy and halophenyl groups designate alkyl, alkoxy and phenyl groups as defined herein, which alkyl, alkoxy or phenyl group is substituted one or more times with halo. Thus a trihalomethyl group represents e.g. a trifluoromethyl group, a trichloromethyl group, and similar trihalo-substituted alkyl groups, and a trihaloalkoxy group designates e.g. a trifluoromethoxy group, a trichloromethoxy, and similar trihalosubstituted alkoxy groups. Preferred haloalkyl groups of the invention include trihalogenmethyl, preferably CF₃, and preferred trihaloalkoxy groups of the invention include trihalomethoxy, preferably —OCF₃.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention a cycloalkyl-alkyl group designates a cycloalkyl group as defined above, which cycloalkyl group is substituted on an alkyl group as also defined above. Examples of preferred cycloalkyl-alkyl groups of the invention include cyclopropylmethyl and cyclopropylethyl.

In the context of this invention an alkoxy group designates an “alkyl-O—” group, wherein alkyl is as defined above. Examples of preferred alkoxy groups of the invention include methoxy and ethoxy.

Pharmaceutically Acceptable Salts

The diphenyl urea derivative for use according to the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a diphenyl urea derivative for use according to the invention and its pharmaceutically acceptable acid addition salt.

Examples of pharmaceutically acceptable cationic salts of the diphenyl urea derivative for use according to the invention include, without limitation, the sodium, the potassium, the calcium, the magnesium, the zinc, the aluminium, the lithium, the choline, the lysine, and the ammonium salt, and the like, of the diphenyl urea derivative for use according to the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

The diphenyl urea derivative for use according to the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Methods of Preparation

The diphenyl urea derivative for use according to the invention may be prepared by conventional methods for chemical synthesis, e.g. those described in publications referenced above, and those described in the working examples. The starting materials for the processes described in the present application are known or may readily be prepared by conventional methods from commercially available chemicals.

Also one diphenyl urea derivative for use according to the invention can be converted to another compound of the invention using conventional methods.

The end products of the reactions described herein may be isolated by conventional techniques, e.g. by extraction, crystallisation, distillation, chromatography, etc.

Pharmaceutical Compositions

In another aspect the invention provides novel pharmaceutical compositions comprising a therapeutically effective amount of the diphenyl urea derivative for use according to the invention.

While the diphenyl urea derivative for use according to the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the diphenyl urea derivative for use according to the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the diphenyl urea derivative for use according to the invention, which matrices may be in form of shaped articles, e.g. films or microcapsules.

Alternatively, or concurrently, administration may be by the oral or nasal route or directly to the lungs. In a preferred embodiment, the compounds of this invention may be administered by inhalation. For inhalation therapy the compound may be in a solution useful for administration by liquid aerosol, metered dose inhalers, or in a form suitable for a dry powder inhaler. The dosage administered will be dependent upon the age, health, and weight of the recipient, kind of concurrent treatment, if any, frequency of treatment, and the nature of the effect desired.

In a preferred embodiment, the diphenyl urea derivative for use according to the invention may be formulated as aerosols. The formulation of pharmaceutical aerosols is routine to those skilled in the art, see e.g. Sciarra J, in Remington: The Science and Practice of Pharmacy 19TH Edition, 1995, Chapter 95, Mack Publishing Company, Easton. The diphenyl urea derivative for use according to the invention may be formulated as solution aerosols, dispersion or suspension aerosols of dry powders, emulsions or colloid preparations. The aerosol may be delivered using any propellant system known to those skilled in the art. The aerosols may be applied to the upper respiratory tract, for example by nasal inhalation, or to the lower respiratory tract or to both.

In other preferred embodiments of the invention, the diphenyl urea derivative for use according to the invention may be formulated into particulates or micronized to improve bioavailability and digestive absorption. In particular, talniflumate may be formulated and micronized using standard techniques in the art, including the methods discussed by Chaumeil J C, et al., Methods Find. Exp. Clin. Pharmacol. 1998 20 3 211-215. In this process, grinding may be carried out in ball or hammer mills of the customary type. These procedures can also be carried out by micronization in gaseous jet micronizers which have the advantage of not heating the substances to be micronized.

The devices of the present invention may be any device adapted to introduce one or more therapeutic compositions into the upper and/or lower respiratory tract. In some preferred embodiments, the devices of the present invention may be metered-dose inhalers. The devices may be adapted to deliver the therapeutic compositions of the invention in the form of a finely dispersed mist of liquid, foam or powder. The devices may use any propellant system known to those in the art including, but not limited to, pumps, liquefied-gas, compressed gas and the like. Devices of the present invention typically comprise a container with one or more valves throw which the flow of the therapeutic composition travels and an actuator for controlling the flow. Suitable devices for use in the present invention may be seen in, for example, in Remington: The Science and Practice of Pharmacy, op cit.

The diphenyl urea derivative for use according to the invention can be provided alone, or in combination with other agents that modulate a particular pathological process. For example, an agent of the present invention can be administered in combination with anti-asthma agents. In another embodiment, the diphenyl urea derivative for use according to the invention may be administered in combination with expectorants, mucolytics, antibiotics, antihistamines or decongestants. In still another embodiment, the diphenyl urea derivative for use according to the invention may be administered along with a surfactant, a stabilizing agent, an absorption-enhancing agent, a beta adrenoreceptor or purine receptor agonist or a flavoring or other agent that increases the palatability of the compositions. As an example, compositions of the invention may contain, in addition to the active substance, an expectorant such as guaifenesin, a stabilizing agent such as cyclodextran and/or an absorption-enhancing agent such as chitosan. Any such agents may be used in the compositions of the invention.

As used herein, two or more active ingredients are said to be administered in combination when the agents are administered simultaneously or are administered independently in a fashion such that the agents will act at the same time.

The diphenyl urea derivative for use according to the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The diphenyl urea derivative for use according to the present invention can be administered in a wide variety of oral and parenteral dosage forms. It will be obvious to those skilled in the art that the following dosage forms may comprise, as the active component, either the diphenyl urea derivative for use according to the invention or a pharmaceutically acceptable salt of such compounds.

For preparing pharmaceutical compositions from the diphenyl urea derivative for use according to the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The diphenyl urea derivative for use according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the diphenyl urea derivative for use according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Further details on techniques for formulation and administration may be found in the latest edition of Remington's Pharmaceutical Sciences (Mack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Biological Activity

The diphenyl urea derivative for use according to the invention are shown to be potent potassium channel activators. Therefore, in one aspect of the invention the diphenylurea derivatives may find use as therapeutic agents in the treatment, prevention or alleviation of a disease or a disorder or a condition that is responsive to modulation of BK_{Ca} channels.

In a preferred embodiment the disease, disorder or condition responsive to modulation of BK_{Ca} channels is a cardiovascular disease, an obstructive or inflammatory airway disease, urinary incontinence, psychosis, epilepsy or pain.

In a preferred embodiment the disease, disorder or condition responsive to modulation of BK_{Ca} channels is a cardiovascular disease. In an even more preferred embodiment the cardiovascular disease is atherosclerosis, ischemia/reperfusion, hypertension, restenosis, arterial inflammation, myocardial ischaemia and ischaemic heart disease.

In another preferred embodiment the disease, disorder or condition responsive to modulation of BK_{Ca} channels is an obstructive or inflammatory airway disease. In an even more preferred embodiment the obstructive or inflammatory airway disease is an airway hyperreactivity, a pneumoconiosis such as aluminosis, anthracosis, asbestosis, chalicosis, ptilosis, siderosis, silicosis, tabacosis and byssinosis, a chronic obstructive pulmonary disease (COPD), bronchitis, excerbation of airways hyperreactivity or cystic fibrosis, or cough including chronic cough. In a most preferred embodiment the obstructive airway disease is chronic obstructive pulmonary disease (COPD).

In a most preferred embodiment the disease, disorder or condition is an obstructive or inflammatory airway disease, in particular chronic obstructive pulmonary disease (COPD), and the diphenyl urea derivative for use according to the invention is N-(3,5-Dichlorophenyl)-N′-[3-(1H-tetrazol-5-yl)-3′-trifluoromethyl-biphenyl-4-yl]-urea; or a pharmaceutically acceptable salt thereof.

In a third preferred embodiment the disease, disorder or condition responsive to modulation of BK_{Ca} channels is urinary incontinence.

In a fourth preferred embodiment the disease, disorder or condition responsive to modulation of BK_{Ca} channels is psychosis.

In a fifth preferred embodiment the disease, disorder or condition responsive to modulation of BK_{Ca} channels is epilepsy.

In a sixth preferred embodiment the disease, disorder or condition responsive to modulation of BK_{Ca} channels is pain.

However, the diphenylurea derivatives of the invention may also be well suited for facilitating the transport of therapeutic substances across the blood-brain barrier, and in particular for facilitating the transvascular delivery of chemotherapeutic agents and viral particles to tumour cells and other abnormal brain tissues.

Therefore, in another aspect, the invention relates to the use of a diphenylurea derivative of the invention as a facilitating agent, useful for increasing the blood-brain barrier permeability, and thus capable of facilitating transport of a therapeutic substance across the blood-brain barrier, including the blood-tumour barrier found in brain tumours.

In a preferred embodiment of this aspect the diphenylurea derivative of the invention is used for facilitating agents to an abnormal brain region of brain tissue physiologically affected by injury, trauma, infection, stroke, or ischemia. This abnormal brain region is a region of benign or malignant tumor tissue or other neoplastic diseases or conditions. The malignant tumor may in particular be a glioma, glioblastoma, oligodendroglioma, astrocytoma, ependymoma, primitive neuroectodermal tumor, atypical meningioma, malignant meningioma, neuroblastoma, sarcoma, melanoma, lymphoma, or carcinoma.

When used as a facilitating agent, the diphenylurea derivative of the invention may be co-administered with the therapeutic agent by any appropriate route, in any convenient way. Preferably, the facilitating agent is administered simultaneously (i.e. contemporaneously or concurrently), or substantially simultaneously (i.e. within about one hour, preferably within 30 minutes, even more preferred within 15 minutes) with the therapeutic agent.

The agents for use according to the invention, i.e. both the facilitating agent and the therapeutic agent, may be administered by any appropriate route, by which the agent is delivered to the blood stream. This is preferably done by intravenous, intramuscular or intra-arterial injection or infusion.

The therapeutic agent for use according to the invention may be any agent or drug. However, preferred therapeutic agents or drugs for use according to the invention are antineoplastic agents, chemotherapeutic agents, cytotoxic agents, DNA expression vectors, proteins, oligonucleotides, nucleotide analogs, antimicrobial agents, interferons, cytokines, cytokine agonists, cytokine antagonists, immunotoxins, immunosuppressants, boron compounds, monoclonal antibodies, adrenergic agents, anticonvulsants, ischemia-protective agents, anti-trauma agents, anticancer chemotherapeutic agents and diagnostic agents.

Preferred chemotherapeutic agents for use according to the invention include:

alkylating agents like the nitrogen mustards (e.g. mechlorethamine, cyclophosphamide, ifosamide, melphalan and chlorambucil), ethylenimines and methylmelamines (e.g. hexamethylmelamine and thiotepa), alkyl sulfonates (e.g. busulfan), nitrosoureas (e.g. carmustine (BCNU), lomustine (CCNU), semustine (methyl-CCNU) and streptozocin), triazenes (e.g. dacarbazine (DTIC));

antimetabolites like folic acid analogs (e.g. methotrexate), pyrimidine analogs (e.g. fluorouracil, floxuridine and cytarabine), purine analogs and related inhibitors (e.g. mercaptopurine, thioguanine and pentostatin); and

natural antimitotic products like vinca alkaloids (e.g. vinblastine and vincristine), epipodophyllotoxins (e.g. etoposide and teniposide), antibiotics (e.g. dactinomycin, daunorubicin, doxorubicin, bleomycin, plicamycin and mitomycin), enzymes (e.g. L-asparaginase), a platinum coordination complex (e.g. cisplatin and carboplatin) and biological response modifiers like the interferons (e.g. interferon-α).

In another preferred embodiment the DNA expression vector is a viral vector, preferably an adenovirus-derived vector or herpes simplex virus-derived vector.

In yet another preferred embodiment the diagnostic agent for use according to the invention may in particular be an imaging or contrast agent, and it may in particular be a radioactively labelled substance, a gallium-labelled substance, or a contrast agent selected from the group consisting of ferrous magnetic, fluorescent, luminescent, and iodinated contrast agents.

When used as a facilitating agent, the diphenylurea derivative of the invention may preferably be co-administered with the therapeutic agent for targeting regions of brain tissue physiologically directly affected by a physical or biochemical injury, for example Alzheimer's disease, Parkinson's disease, Parkinsonism, trauma, infection, stroke, brain ischemia, or regions of neoplastic growth within the brain, such as benign or malignant brain tumour tissues.

Methods of Therapy

In yet another aspect the invention provides methods of treatment, prevention or alleviation of an obstructive or inflammatory airway disease, urinary incontinence, psychosis, epilepsy or pain in a living animal body, including a human, which method comprises the step of administering to such a living animal body in need thereof, a therapeutically effective amount of the diphenyl urea derivative of the invention.

In a preferred embodiment the disease, disorder or condition is of an obstructive or inflammatory airway disease. In a more preferred embodiment the diphenyl urea derivative for use in the method of the invention is N-(3,5-Dichlorophenyl)-N′-[3-(1H-tetrazol-5-yl)-3′-trifluoromethyl-biphenyl-4-yl]-urea; or a pharmaceutically acceptable salt thereof.

In another preferred embodiment the invention provides method of increasing the blood-brain barrier permeability in a living animal body, including a human, which method comprises the step of administering to such a living animal body in need thereof, a therapeutically effective amount of a diphenyl urea derivative of the invention.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge. When administered in combination with compounds known in the art for treatment of the diseases, the dose may be reduced.

### BRIEF DESCRIPTION OF THE DRAWINGS

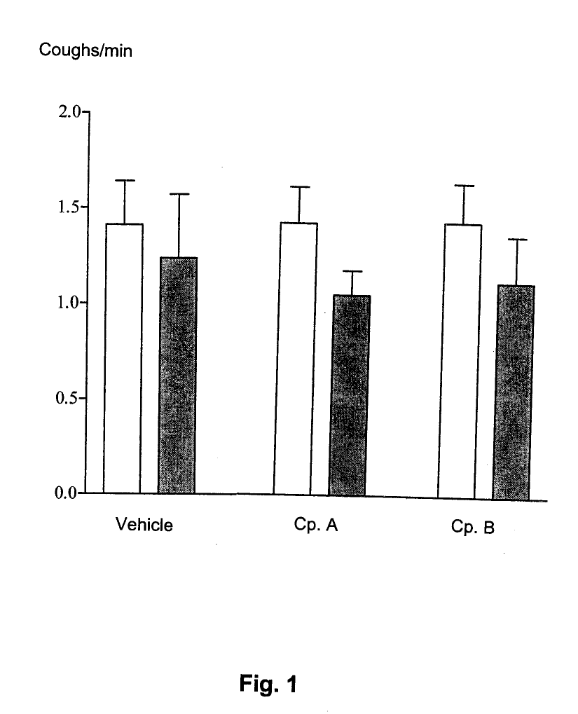

The present invention is further illustrated by reference to the accompanying drawing, in which FIG. 1 shows the effect of Compounds A and B of the invention on the cough reflex in conscious guinea-pigs. Exposure of animals for 10 minutes with citric acid aerosol elicited a reproducible cough response. After a prior exposure to aerosolized test compound (300 μM in nebulizer solution) the cough response to citric acid was greatly reduced.

### EXAMPLES

The invention is further illustrated with reference to the following examples, which are not intended to be in any way limiting to the scope of the invention as claimed.

### Example 1

### Preparative Example

### Intermediate Compounds

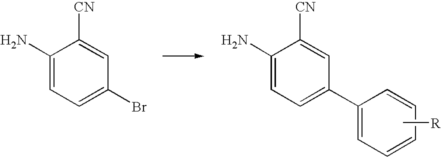

### 4-Amino-4′-trifluoromethyl-biphenyl-3-carbonitrile

To dimethoxyethane (100 mL) and water (50 mL) was added 2-amino-5-bromo-benzonitrile (8.1 g), 4-trifluoromethyl-phenyl-boronic acid (8.6 g) and potassium carbonate (18.7 g), nitrogen was bobbled through the mixture for 10 minutes. Under a nitrogen atmosphere was bis(triphenylphosphine)palladium (II) chloride (0.3 g) added, the reaction mixture was heated at reflux overnight, then cooled to room temperature and added water (150 mL). The mixture was extracted with ethyl acetate, the organic phase was washed with water (50 mL) and brine (50 mL), then dried with magnesium sulfate and evaporated to an oil. The product was purified by column chromatography. Yield 8.36 g of white powder.

Similarly was made:
4-Amino-4′-chloro-biphenyl-3-carbonitrile;
4-Amino-4′-fluoro-biphenyl-3-carbonitrile;
4-Amino-4′-methyl-biphenyl-3-carbonitrile;
4-Amino-4′-trilfluoromethoxy-biphenyl-3-carbonitrile; and
4-Amino-3′-triluoromethyl-biphenyl-3-carbonitrile.

### Example 2

### Preparative Example

### Intermediate Compounds

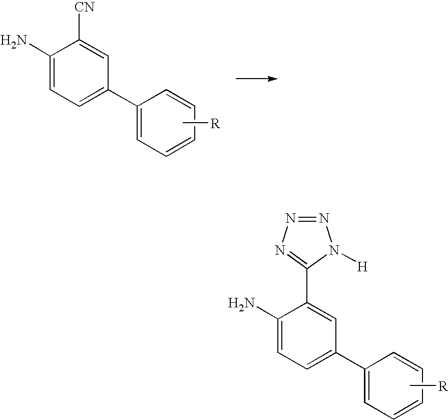

### 3-(1H-Tetrazol-5-yl)-4′-trifluoromethyl-biphenyl-4-ylamine

4-Amino-4′-trifluoromethyl-biphenyl-3-carbonitrile (8.3 g) was dissolved in toluene (100 mL), to the solution was added sodium azide (3.1 g) and triethylammonium chloride (6.6 g). The reaction mixture was heated at 60-62° C. overnight, then cooled to room temperature and added water (40 mL), then hydrochloric acid (4 M; 13 mL) was added until pH=1. The product precipitated and was isolated by filtration, the precipitate was washed with cold water and dried on the filter by sucking air through the compound. Yield 10.2 g of white powder.

Similarly was made:
4′-Chloro-3-(1H-tetrazol-5-yl)-biphenyl-4-ylamine;
4′-Fluoro-3-(1H-tetrazol-5-yl)-biphenyl-4-ylamine;
4′-Methyl-3-(1H-tetrazol-5-yl)-biphenyl-4-ylamine;
3-(1H-tetrazol-5-yl)-4′-trifluoromethoxy-biphenyl-4-ylamine; and
3-(1H-tetrazol-5-yl)-3′-trifluoromethyl-biphenyl-4-ylamine.

### Example 3

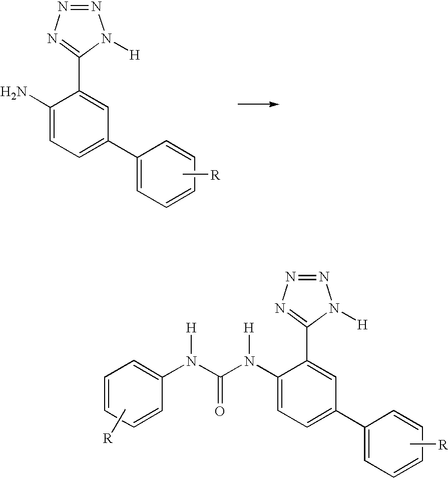

### N-(4-Chloro-3-trifluoromethyl-phenyl)-N′-[3-(1H-tetrazol-5-yl)-4′-trifluoromethyl-biphenyl-4-yl]-urea (Compound 3-1)

3-(1H-Tetrazol-5-yl)-4′-trifluoromethyl-biphenyl-4-ylamine (0.5 g) and 4-chloro-3-trifluoromethyl-phenyl isocyanate (0.4 g) in toluene (15 mL) was stirred at room temperature for two days. The reaction mixture was evaporated to an oil, the oil was dissolved in acetone and filtrated through Celite, the filtrate was added water, the product precipitated and was isolated by filtration. Yield 0.6 g. Mp. 226-228° C.

Similarly was made:
N-(3-Trifluoromethyl-phenyl)-N′-[4′-chloro-3-(1H-tetrazol-5-yl)-biphenyl-4-yl]-urea (Compound 3-2): Mp. 253-254° C.;
N-(4-Chloro-3-trifluoromethyl-phenyl)-N′-[4′-chloro-3-(1H-tetrazol-5-yl)-biphenyl-4-yl]-urea (Compound 3-3): Mp. 242-243° C.;
N-(3,5-Dichloro-phenyl)-N′-[4′-chloro-3-(1H-tetrazol-5-yl)-biphenyl-4-yl]-urea (Compound 3-4): Mp. 231-234°;
N-(3,5-Difluoro-phenyl)-N′-[4′-chloro-3-(1H-tetrazol-5-yl)-biphenyl-4-yl]-urea (Compound 3-5): Mp. 250-251°;
N-(3,5-Dichloro-phenyl)-N′-[3-(1H-tetrazol-5-yl)-4′-trifluoromethyl-biphenyl-4-yl]-urea (Compound 3-6): Mp. 226-230°;
N-(3,5-Difluoro-phenyl)-N′-[3-(1H-tetrazol-5-yl)-4′-trifluoromethyl-biphenyl-4-yl]-urea (Compound 3-7): Mp. 245-247° C.;
N-(3-Trifluoromethyl-phenyl)-N′-[4′-fluoro-3-(1H-tetrazol-5-yl)-biphenyl-5-yl]-urea (Compound 3-8): Mp. 256-258° C.;
N-(4-Chloro-3-trifluoromethyl-phenyl)-N′-[4′-fluoro-3-(1H-tetrazol-5-yl)-biphenyl-4-yl]-urea (Compound 3-9): Mp. 247-249° C.;
N-(3,5-Dichloro-phenyl)-N′-[4′-fluoro-3-(1H-tetrazol-5-yl)-biphenyl-4-yl]-urea (Compound 3-10): Mp. 241-243° C.;
N-(3,5-Difluoro-phenyl)-N′-[4′-fluoro-2-(1H-tetrazol-5-yl)-biphenyl-4-yl]-urea (Compound 3-11): Mp. 255-256° C.;
N-(4-Fluoro-3-trifluoromethyl-phenyl)-N′-[3-(1H-tetrazol-5-yl)-4′-trifluoromethyl-biphenyl-4-yl]-urea (Compound 3-12): Mp. 247-249° C. (subl.);
N-(3,5-Bis-trifluoromethyl-phenyl)-N′-[3-(1H-tetrazol-5-yl)-4′-trifluoromethyl-biphenyl-4-yl]-urea (Compound 3-13): Mp. 246-248° C.;
N-(3,5-Bis-trifluoromethyl-phenyl)-N′-[4′-chloro-3-(1H-tetrazol-5-yl)-biphenyl-4-yl]-urea (Compound 3-14): Mp. 230-233° C.;
N-(4-Fluoro-3-trifluoromethyl-phenyl)-N′-[4′-chloro-3-(1H-tetrazol-5-yl)-biphenyl-4-yl]-urea (Compound 3-15): Mp. 243-245° C.;
N-(3,5-Bis-trifluoromethyl-phenyl)-N′-[4′-fluoro-3-(1H-tetrazol-5-yl)-biphenyl-4-yl]-urea (Compound 3-16): Mp. 251-253° C.;
N-(4-Fluoro-3-trifluoromethyl-phenyl)-N′-[4′-fluoro-3-(1H-tetrazol-5-yl)-biphenyl-4-yl]-urea (Compound 3-17): Mp. 253-254° C.;
N-(2-Trifluoromethyl-phenyl)-N′-[3-(1H-tetrazol-5-yl)-4′-trifluoromethyl-biphenyl-4-yl]-urea (Compound 3-18): Mp. 240-243° C.;
N-(2-Trifluoromethyl-phenyl)-N′-[4′-fluoro-3-(1H-tetrazol-5-yl)-biphenyl-4-yl]-urea (Compound 3-19): Mp. 256-258° C.;
N-(2-Bromo-phenyl)-N′-[3-(1H-tetrazol-5-yl)-4′-trifluoromethyl-biphenyl-4-yl]-urea (Compound 3-20): Mp. 242-243° C.;
N-(2-Trifluoromethyl-phenyl)-N′-[4′-chloro-3-(1H-tetrazol-5-yl)-biphenyl-4-yl]-urea (Compound 3-21): Mp. 290-292° C.;
N-(2-Bromo-phenyl)-N′-[4′-chloro-3-(1H-tetrazol-5-yl)-biphenyl-4-yl]-urea (Compound 3-22): Mp. 255-256° C.;
N-(2-Bromo-phenyl)-N′-[4′-fluoro-3-(1H-tetrazol-5-yl)-biphenyl-4-yl]-urea (Compound 3-23): Mp. 256-258° C.;
N-(2-Fluoro-phenyl)-N′-[3-(1H-tetrazol-5-yl)-4′-trifluoromethyl-biphenyl-4-yl]-urea (Compound 3-24): Mp. 251-252° C.;
N-(2-Fluoro-phenyl)-N′-[4′-chloro-3-(1H-tetrazol-5-yl)-biphenyl-4-yl]-urea (Compound 3-25): Mp. 257-259° C.;
N-(2-Fluoro-phenyl)-N′-[4′-fluoro-3-(1H-tetrazol-5-yl)-biphenyl-4-yl]-urea (Compound 3-26): Mp. 263-264° C.;
N-(2-Fluoro-phenyl)-N′-[4′-methyl-3-(1H-tetrazol-5-yl)-biphenyl-4-yl]-urea (Compound 3-27): Mp. 260-262° C.;
N-(2-Chloro-phenyl)-N′-[4′-methyl-3-(1H-tetrazol-5-yl)-biphenyl-4-yl]-urea (Compound 3-28): Mp. 261-263° C.;
N-(2-Bromo-phenyl)-N′-[4′-methyl-3-(1H-tetrazol-5-yl)-biphenyl-4-yl]-urea (Compound 3-29): Mp. 255-257° C.;
N-(2-Trifluoromethyl-phenyl)-N′-[4′-methyl-3-(1H-tetrazol-5-yl)-biphenyl-4-yl]-urea (Compound 3-30): Mp. 259-261° C.;
N-(2-Chloro-phenyl)-N′-[3-(1H-tetrazol-5-yl)-4′-trifluoromethyl-biphenyl-4-yl]-urea (Compound 3-31): Mp. 254-255° C. (subl.);
N-(2-Chloro-phenyl)-N′-[4′-chloro-3-(1H-tetrazol-5-yl)-biphenyl-4-yl]-urea (Compound 3-32): Mp. 255-257° C. (subl.);
N-(2-Chloro-phenyl)-N′-[4′-fluoro-3-(1H-tetrazol-5-yl)-biphenyl-4-yl]-urea (Compound 3-33): Mp. 255-257° C. (subl.);
N-(3,5-Dichloro-phenyl)-N′-[4′-methoxy-3-(1H-tetrazol-5-yl)-biphenyl-4-yl]-urea (Compound 3-34): Mp. 200-201° C.;
N-(3,5-Difluoro-phenyl)-N′-[3-(1H-tetrazol-5-yl)-4′-trifluoromethoxy-biphenyl-4-yl]-urea (Compound 3-35): Mp. 238-241° C.;
N-(3,5-Dichloro-phenyl)-N′-[3-(1H-tetrazol-5-yl)-4′-trifluoromethoxy-biphenyl-4-yl]-urea (Compound 3-36): Mp. 224-225° C.;
N-(3,5-Bis-trifluoromethyl-phenyl)-N′-[3-(1H-tetrazol-5-yl)-4′-trifluoromethoxy-biphenyl-4-yl]-urea (Compound 3-37): Mp. 238-240° C. (subl.);
N-(3,5-Difluoro-phenyl)-N′-[3-(1H-tetrazol-5-yl)-3′-trifluoromethyl-biphenyl-4-yl]-urea (Compound 3-38): Mp. 255-257° C.;
N-(3,5-Dichloro-phenyl)-N′-[3-(1H-tetrazol-5-yl)-3′-trifluoromethyl-biphenyl-4-yl]-urea (Compound 3-39): Mp. 236-239° C. (subl.);
N-(3,5-Bis-trifluoromethyl-phenyl)-N′-[3-(1H-tetrazol-5-yl)-3′-trifluoromethyl-biphenyl-4-yl]-urea (Compound 3-40): Mp. 250-252° C.;
N-(3,5-Difluoro-phenyl)-N′-[4′-methoxy-3-(1H-tetrazol-5-yl)-biphenyl-4-yl]-urea (Compound 3-41): Mp. 129-133° C.;
N-(3,5-Bis-trifluoromethyl-phenyl)-N′-[4′-methoxy-3-(1H-tetrazol-5-yl)-biphenyl-4-yl]-urea (Compound 3-42): Mp. 219-221° C.;
N-(3-Bromo-phenyl)-N′-[3-(1H-tetrazol-5-yl)-4′-trifluoromethyl-biphenyl-4-yl]-urea (Compound 3-43): Mp. 203-210° C. (subl.);
N-(4-Chloro-phenyl)-N′-[3-(1H-tetrazol-5-yl)-4′-trifluoromethyl-biphenyl-4-yl]-urea (Compound 3-44): Mp. 232-234° C.; and
N-(4-Fluoro-3-trifluoromethyl-phenyl)-N′-[3-(1H-tetrazol-5-yl)-3′-trifluoromethyl-biphenyl-4-yl]-urea (Compound 3-45): Mp. 254-255° C.

### Example 4

### Electrophysiological Determination

In this experiment we investigated the influence of the compounds of the invention on the membrane currents when determined electrophysiologically on Xenopus Oocytes capable of expressing human BK channels, and the current through the channels is recorded using the classical two-electrode voltage clamp technique.

Initially Compounds 3-7, 3-13, 3-16, 3-37, 3-38, 3-39 and 3-42 were subjected to this determination at concentrations of 0.3 μM of test compound, and they caused an increase of BK current relative to the basal current in the range of 200 to 600%, indication a potent BK activating activity.

### Example 5

### Effect on Citric Acid-Induced Cough

In this experiment we investigated the influence of the compounds of the invention on fit of coughing induced by citric acid.

The cough model is based on guinea-pigs that have been pre-screened to assess their cough response to a 10 minutes exposure of aerosolised 0.35 M citric acid. Animals that do not cough were excluded from the study. The guinea-pigs are then ranked with regard to their tussive response to citric acid and divided into groups so that the mean number of coughs per group is similar in the vehicle and drug treated groups.

Cough is detected both by pressure change and by sound and recorded using a chart recorder. All animals received Terbutaline (0.05 mg/kg i.p.) 10 minutes prior to challenge with citric acid to alleviate any bronchoconstriction that may occur. These animals were allowed to recover for 1 week. Following this rest period the study was initiated and animals received aerosolised vehicle (1% DMSO) or test compound at 300 μM in 1% DMSO in saline for 20 minutes prior to exposure to citric acid. After dosing, the animals were monitored and exposed to 0.35 M citric acid for 10 minutes, and the number of coughs recorded.

Initially N-(3,5-Dichlorophenyl)-N′-[3-(1H-tetrazol-5-yl)-3′-trifluoromethyl-bi-phenyl-4-yl]-urea (Compound A (3-39)) and N-(3,5-Dichlorophenyl)-N′-[4-bromo-2-(1H-tetrazol-5-yl)phenyl]urea (Compound B) were subjected to this determination, and the results of these determinations are presented in FIG. 1.

1. A method of treatment, prevention or alleviation of an obstructive or inflammatory airway disease, urinary incontinence, psychosis, epilepsy or pain in a living animal body, including a human, which method comprises the step of administering to such a living animal body in need thereof, a therapeutically effective amount of a diphenyl urea derivative represented by Formula I 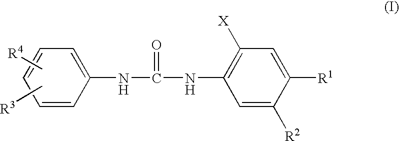
or a pharmaceutically acceptable salt thereof, wherein
X represents hydroxy, carboxy, a tetrazolyl group, an oxadiazolyl group or a triazolyl group;
R¹ represents hydrogen, alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, N-alkyl-piperazinyl-carbonyl, carbamoyl-N-alkyl-piperazine, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride, phenyl, naphthyl, pyridyl, furyl or thienyl; or
R¹ represents phenyl substituted once or twice with alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, carbamoyl-N-alkyl-piperazine, N-alkyl-piperazinyl-carbonyl, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride;
R² represents hydrogen, halo, haloalkyl, alkoxy, alkoxy-carbonyl, nitro, halophenyl, haloalkyl-phenyl or haloalkoxy-phenyl;
R³ represents hydrogen, alkyl, halo, haloalkyl, nitro, hydroxy, alkoxy, haloalkoxy, carboxy, alkyl-carbonyl, alkoxy-carbonyl, amino-carbonyl, benzoyl, acetyl, phenyl, pyridyl; or phenyl substituted with alkyl, halo or haloalkyl; and
R⁴ represents hydrogen, alkyl, halo, haloalkyl, nitro, hydroxy, alkoxy, phenyl, pyridyl, or phenyl substituted with haloalkyl; or
R³ and R⁴ together with the phenyl to which they are attached form a naphthyl group;
for the manufacture of a pharmaceutical composition for the treatment, prevention or alleviation of a disease or a disorder or a condition of a mammal, including a human, which disease or disorder or condition is a cardiovascular disease, an obstructive or inflammatory airway disease, urinary incontinence, psychosis, epilepsy or pain.

2. The method according to claim 1, wherein X represents hydroxy, carboxy, a tetrazolyl group, an oxadiazolyl group or a triazolyl group.

3. The method according to claim 2, wherein X represents a tetrazolyl group, an oxadiazolyl group or a triazolyl group.

4. The method according to claim 1, wherein
R¹ represents hydrogen, alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, N-alkyl-piperazinyl-carbonyl, carbamoyl-N-alkyl-piperazine, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride, phenyl, naphthyl, pyridyl, furyl or thienyl; or
R¹ represents phenyl substituted once or twice with alkyl, halo, haloalkyl, hydroxy, alkoxy, haloalkoxy, nitro, cyano, amino, N-phenyl-amino, N-benzoyl-amino, alkyl-carbonyl-amino, carboxy, alkyl-carbonyl, cycloalkyl-carbonyl, alkoxy-carbonyl, amino-carbonyl (carbamoyl), N-alkyl-amino-carbonyl, N,N-dialkyl-amino-carbonyl, N-phenyl-amino-carbonyl, N-benzoyl-amino-carbonyl, N,N-dialkyl-amino-carbonyl, N-alkyl-N-acetic acid amino-carbonyl, N-carboxy-alkyl-amino-carbonyl (N-acetic acid carboxamide), anilino-carbonyl, pyrrolidinyl-carbonyl, piperidinyl-carbonyl, piperazinyl-carbonyl, carbamoyl-N-alkyl-piperazine, N-alkyl-piperazinyl-carbonyl, N,N-dialkyl acryl-amide, amino-carbonyl-alkyl, N-alkyl-amino-carbonyl-alkyl, N,N-dialkyl-amino-carbonyl-alkyl, sulfamoyl, N-alkyl-sulfamoyl, N,N-dialkylsulfamoyl or sulfonamido-N-alkyl-piperazinium chloride.

5. The method according to claim 4, wherein
R¹ represents hydrogen, alkyl, halo, haloalkyl, hydroxy, alkoxy, haloalkoxy, nitro, amino, alkyl-carbonyl-amino, N-phenyl-amino, N-benzoyl-amino, N,N-dialkyl acryl-amide, N,N-dialkyl-amino-carbonyl, N,N-dialkyl-amino-carbonyl-alkyl, alkoxy-carbonyl, phenyl, naphthyl, pyridyl, furyl or thienyl; or
R¹ represents phenyl substituted with alkyl, halo, haloalkyl, haloalkoxy, nitro, amino, carboxy, alkoxy-carbonyl, amino-carbonyl (carbamoyl), N,N-dialkyl-amino-carbonyl, N,N-dialkyl-amino-carbonyl-alkyl, N-phenyl-amino-carbonyl, N-alkyl-N-acetic acid amino-carbonyl, N-acetic acid-amino-carbonyl, anilino-carbonyl, piperidinyl-carbonyl, carbamoyl-N-alkyl-piperazine, N,N-dialkyl-sulfamoyl or sulfonamido-N-alkyl-piperazinium chloride.

6. The method according to claim 1, wherein
R² represents hydrogen, halo, haloalkyl, alkoxy, alkoxy-carbonyl, nitro, halophenyl, haloalkyl-phenyl or haloalkoxy-phenyl.

7. The method according to claim 1, wherein
R³ represents hydrogen, alkyl, halo, haloalkyl, nitro, hydroxy, alkoxy, haloalkoxy, carboxy, alkyl-carbonyl, alkoxy-carbonyl, amino-carbonyl, benzoyl, phenyl, pyridyl, or phenyl substituted with alkyl, halo, haloalkyl or haloalkoxy
R⁴ represents hydrogen, alkyl, halo, haloalkyl, nitro, hydroxy, alkoxy, haloalkoxy, carboxy, alkyl-carbonyl, alkoxy-carbonyl, amino-carbonyl, benzoyl, phenyl, pyridyl, or phenyl substituted with alkyl, halo, haloalkyl or haloalkoxy; or
R³ and R⁴ together with the phenyl to which they are attached form a naphthyl group.

8. The method according to claim 1, wherein the diphenyl urea derivative is represented by Formula II 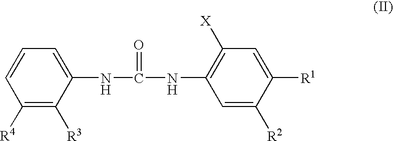
or a pharmaceutically acceptable salt thereof, wherein
X, R¹ and R² are as defined in claim 1, and
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl or pyridyl; or phenyl substituted with alkyl, halo or haloalkyl; and
R⁴ represents hydrogen, alkyl, halo, haloalkyl, nitro, alkoxy, or phenyl; or phenyl substituted with haloalkyl; or
R³ and R⁴ together with the phenyl to which they are attached form a naphthyl group.

9. The method according to claim 8, wherein
X represents a tetrazolyl group, an oxadiazolyl group or a triazolyl group;
R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or
R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, piperidin-1-yl-carbonyl, amino-carbonyl-N-alkyl-piperazine, N,N-dialkylsulfamoyl or sulfonamido-N-alkyl-piperazinium chloride; and
R² represents hydrogen, halo, alkoxy, alkoxy-carbonyl, nitro, halophenyl, haloalkyl-phenyl or haloalkoxy-phenyl;
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen; alkyl; halo; haloalkyl; nitro; alkoxy; phenyl or phenyl substituted with haloalkyl; or
R³ and R⁴ together with the phenyl to which they are attached form a naphthyl group.

10. The method according to claim 9, wherein the diphenyl urea derivative is
N-(2-Nitrophenyl)-N′-[4-bromo-2-(1H-tetrazol-5-yl)phenyl]urea;
N-(2-Methylphenyl)-N′-[4-bromo-2-(1H-tetrazol-5-yl)phenyl]urea;
N-(2-Ethylphenyl)-N′-[4-bromo-2-(1H-tetrazol-5-yl)phenyl]urea;
N-(2-Trifluoromethylphenyl)-N′-[4-bromo-2-(1H-tetrazol-5-yl)phenyl]urea;
N-(2-Bromophenyl)-N′-[4-bromo-2-(1H-tetrazol-5-yl)phenyl]urea;
N-(2-Chlorophenyl)-N′-[4-bromo-2-(1H-tetrazol-5-yl)phenyl]urea;
N-(2-Fluorophenyl)-N′-[4-bromo-2-(1H-tetrazol-5-yl)phenyl]urea;
N-(2-Trifluoromethyl-phenyl)-N′-[3-(1H-tetrazol-5-yl)-4′-trifluoromethyl-biphenyl-4-yl]-urea;
N-(2-Trifluoromethyl-phenyl)-N′-[4′-fluoro-3-(1H-tetrazol-5-yl)-biphenyl-4-yl]-urea;
N-(2-Bromo-phenyl)-N′-[3-(1H-tetrazol-5-yl)-4′-trifluoromethyl-biphenyl-4-yl]-urea;
N-(2-Trifluoromethyl-phenyl)-N′-[4′-chloro-3-(1H-tetrazol-5-yl)-biphenyl-4-yl]-urea;
N-(2-Bromo-phenyl)-N′-[4′-chloro-3-(1H-tetrazol-5-yl)-biphenyl-4-yl]-urea;
N-(2-Bromo-phenyl)-N′-[4′-fluoro-3-(1H-tetrazol-5-yl)-biphenyl-4-yl]-urea;
N-(2-Fluoro-phenyl)-N′-[3-(1H-tetrazol-5-yl)-4′-trifluoromethyl-biphenyl-4-yl]-urea;
N-(2-Fluoro-phenyl)-N′-[4′-chloro-3-(1H-tetrazol-5-yl)-biphenyl-4-yl]-urea;
N-(2-Fluoro-phenyl)-N′-[4′-fluoro-3-(1H-tetrazol-5-yl)-biphenyl-4-yl]-urea;
N-(2-Fluoro-phenyl)-N′-[4′-methyl-3-(1H-tetrazol-5-yl)-biphenyl-4-yl]-urea;
N-(2-Chloro-phenyl)-N′-[4′-methyl-3-(1H-tetrazol-5-yl)-biphenyl-4-yl]-urea;
N-(2-Bromo-phenyl)-N′-[4′-methyl-3-(1H-tetrazol-5-yl)-biphenyl-4-yl]-urea;
N-(2-Trifluoromethyl-phenyl)-N′-[4′-methyl-3-(1H-tetrazol-5-yl)-biphenyl-4-yl]-urea;
N-(2-Chloro-phenyl)-N′-[3-(1H-tetrazol-5-yl)-4′-trifluoromethyl-biphenyl-4-yl]-urea;
N-(2-Chloro-phenyl)-N′-[4′-chloro-3-(1H-tetrazol-5-yl)-biphenyl-4-yl]-urea;
N-(2-Chloro-phenyl)-N′-[4′-fluoro-3-(1H-tetrazol-5-yl)-biphenyl-4-yl]-urea;
N-(2-Chloro-phenyl)-N′-[4′-(piperidin-1-yl-carbonyl)-3-(1H-tetrazol-5-yl)-biphenyl-4-yl]urea;
N-(2-Trifluoromethyl)-N′-[4′-(N″,N″-dimethyl-amino-carbonyl)-3-(1H-tetrazol-5-yl)-biphenyl-4-yl]urea;
N-(1-Naphthyl)-N′-[4-bromo-2-(1H-tetrazol-5-yl)phenyl]urea;
N-(1-Naphthyl)-N′-[4′-(N″,N″-dimethyl-amino-carbonyl)-3-(1H-tetrazol-5-yl)-biphenyl-4-yl]urea;
or a pharmaceutically acceptable salt thereof.

11. The method according to claim 1, wherein the diphenyl urea derivative is represented by Formula III 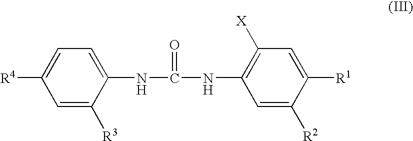
or a pharmaceutically acceptable salt thereof, wherein
X, R¹ and R² are as defined in claim 1, and
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy, phenyl or phenyl substituted with haloalkyl.

12. The method according to claim 11, wherein
X represents a tetrazolyl group, an oxadiazolyl group or a triazolyl group;
R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or
R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and
R² represents hydrogen, halo, alkoxy, alkoxy-carbonyl, nitro, halophenyl, haloalkyl-phenyl or haloalkoxy-phenyl;
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy, phenyl or phenyl substituted with haloalkyl.

13. The method according to claim 12, wherein the diphenyl urea derivative is
N-(2-Chloro-4-trifluoromethylphenyl)-1-[4-bromo-2-(1H-tetrazol-5-yl)phenyl]urea;
N-(4-Biphenyl)-N′-(2-(1H-tetrazol-5-yl)phenyl)urea;
N-(4-Biphenyl)-N′-(5-chloro-2-(1H-tetrazol-5-yl)phenyl)urea;
N-(4-Trifluoromethylphenyl-N′-[4-bromo-2-(1H-tetrazol-5-yl)phenyl]urea;
N-(4-Bromophenyl)-N′-[4-bromo-2-(1H-tetrazol-5-yl)phenyl]urea;
N-(4-Methylphenyl)-N′-[4-bromo-2-(1H-tetrazol-5-yl)phenyl]urea;
N-(4-[2-Propyl]phenyl)-N′-[4-bromo-2-(1H-tetrazol-5-yl)phenyl]urea;
N-(4-Methoxyphenyl)-N′-[4-bromo-2-(1H-tetrazol-5-yl)phenyl]urea;
N-(4-Ethoxyphenyl)-N′-[4-bromo-2-(1H-tetrazol-5-yl)phenyl]urea;
N-(4-Nitrophenyl)-N′-[4-bromo-2-(1H-tetrazol-5-yl)phenyl]urea;
N-(4-Chloro-phenyl)-N′-[3-(1H-tetrazol-5-yl)-4′-trifluoromethyl-biphenyl-4-yl]-urea;
N-(4-Chloro-3-trifluoromethyl-phenyl)-N′-[4′-(N′,N″-dimethyl-amino-carbonyl)-3-(1H-tetrazol-5-yl)-biphenyl-4-yl]urea;
N-(4-Methoxyphenyl)-N′-[4′-(N″,N″-dimethyl-amino-carbonyl)-3-(1H-tetrazol-5-yl)-biphenyl-4-yl]urea;
or a pharmaceutically acceptable salt thereof.

14. The method according to claim 1, wherein the diphenyl urea derivative is represented by Formula IV 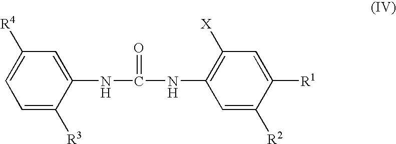
or a pharmaceutically acceptable salt thereof, wherein
X, R¹ and R² are as defined in claim 1, and
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy, phenyl; or phenyl substituted with haloalkyl.

15. The method according to claim 14, wherein
X represents hydroxy or carboxy;
R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or
R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and
R² represents hydrogen, halo, haloalkyl, alkoxy, alkoxy-carbonyl, nitro, halophenyl, haloalkyl-phenyl or haloalkoxy-phenyl;
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy, phenyl; or phenyl substituted with haloalkyl.

16. The method according to claim 15, wherein
X represents hydroxy or carboxy;
R¹ represents hydrogen, halo, nitro, amino, N-phenyl-amino, N-benzoyl-amino, alkyl-carbonyl-amino or N-benzoyl-amino;
R² represents hydrogen, halo, haloalkyl or nitro;
R³ represents hydrogen, alkyl, halo, haloalkyl, nitro, hydroxy or alkoxy; and
R⁴ represents hydrogen, halo, haloalkyl or nitro.

17. The method according to claim 16, wherein the diphenyl urea derivative is
1,3-Bis-(2-hydroxy-5-trifluoromethyl-phenyl)-urea;
or a pharmaceutically acceptable salt thereof.

18. The method according to claim 1, wherein the diphenyl urea derivative is represented by Formula V 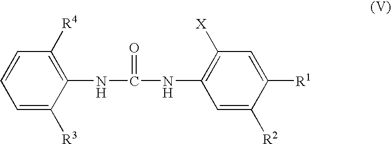
or a pharmaceutically acceptable salt thereof, wherein
X, R¹ and R² are as defined in claim 1, and
R³ represents hydrogen, alkyl, halo, haloalkyl, nitro, hydroxy, alkoxy, phenyl or pyridyl; and
R⁴ represents hydrogen, alkyl, halo, haloalkyl, nitro, hydroxy, alkoxy, phenyl or pyridyl.

19. The method according to claim 18, wherein
X represents a tetrazolyl group, an oxadiazolyl group or a triazolyl group;
R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or
R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and
R² represents hydrogen, halo, alkoxy, alkoxy-carbonyl, nitro, halophenyl, haloalkyl-phenyl or haloalkoxy-phenyl;
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy, phenyl; or phenyl substituted with haloalkyl.

20. The method according to claim 1, wherein the diphenyl urea derivative is represented by Formula VI 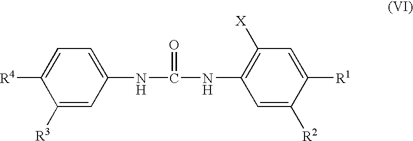
or a pharmaceutically acceptable salt thereof, wherein
X, R¹ and R² are as defined in claim 1, and
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or
R³ represents phenyl substituted with alkyl, halo or haloalkyl; and
R⁴ represents hydrogen, alkyl, halo, haloalkyl, nitro, alkoxy, phenyl; or phenyl substituted with haloalkyl; or
R³ and R⁴ together with the phenyl to which they are attached form a naphthyl group.

21. The method according to claim 20, wherein
X represents a tetrazolyl group, an oxadiazolyl group or a triazolyl group;
R¹ represents hydrogen, halo, hydroxy, alkoxy, nitro, amino, N-phenyl-amino, N-benzoyl-amino, alkyl-carbonyl-amino, N-benzoyl-amino, N,N-dialkyl acryl-amide, alkoxy-carbonyl, phenyl, naphthyl, pyridyl, furyl or thienyl; or
R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, N,N-dialkyl-amino-carbonyl-alkyl, N-alkyl-N-acetic acid amino-carbonyl, anilino-carbonyl, piperidinyl-carbonyl, amino-carbonyl-N-alkyl-piperazine, N,N-dialkyl-sulfamoyl or sulfonamido-N-alkyl-piperazinium chloride;
R² represents hydrogen, halo, alkoxy, alkoxy-carbonyl, nitro, halophenyl, haloalkyl-phenyl or haloalkoxy-phenyl;
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy or phenyl; or phenyl substituted with haloalkyl; or
R³ and R⁴ together with the phenyl to which they are attached form a naphthyl group.

22. The method according to claim 21, wherein the diphenyl urea derivative is
N-(3,4-Dichlorophenyl)-N′-[4-bromo-2-(1H-tetrazol-5-yl)phenyl]urea;
N-(4-Methyl-3-nitrophenyl)-N′-[4-bromo-2-(1H-tetrazol-5-yl)phenyl]urea;
N-(4-Chloro-3-trifluoromethylphenyl)-N′-[4-bromo-2-(1H-tetrazol-5-yl)phenyl]urea;
N-(4-Chloro-3-trifluoromethyl-phenyl)-N′-[3-(1H-tetrazol-5-yl)-4′-trifluoromethyl-biphenyl-4-yl]-urea;
N-(4-Chloro-3-trifluoromethyl-phenyl)-N′-[4′-chloro-3-(1H-tetrazol-5-yl)-biphenyl-4-yl]-urea;
N-(4-Chloro-3-trifluoromethyl-phenyl)-N′-[4′-fluoro-3-(1H-tetrazol-5-yl)-biphenyl-4-yl]-urea;
N-(4-Fluoro-3-trifluoromethyl-phenyl)-N′-[3-(1H-tetrazol-5-yl)-4′-trifluoromethyl-biphenyl-4-yl]-urea;
N-(4-Fluoro-3-trifluoromethyl-phenyl)-N′-[4′-chloro-3-(1H-tetrazol-5-yl)-biphenyl-4-yl]-urea;
N-(4-Fluoro-3-trifluoromethyl-phenyl)-N′-[4′-fluoro-3-(1H-tetrazol-5-yl)-biphenyl-4-yl]-urea;
N-(4-Fluoro-3-trifluoromethyl-phenyl)-N′-[3-(1H-tetrazol-5-yl)-3′-trifluoromethyl-biphenyl-4-yl]-urea;
N-(4-Fluoro-3-chloro-phenyl)-N′-(4′-(N,N-dimethylsulfamoyl)-2-(1H-tetrazol-5-yl)-4-biphenyl)urea; or
N-(4-Chloro-3-trifluoromethyl-phenyl)-N′-(4′-(N,N-dimethylsulfamoyl)-2-(1H-tetrazol-5-yl)-4-biphenyl)urea;
N-(4-Fluoro-3-trifluoromethyl-phenyl)-N′-(4′-(N,N-dimethylsulfamoyl)-2-(1H-tetrazol-5-yl)-4-biphenyl)urea;
N-(4-Chloro-3-trifluoromethyl-phenyl)-N′-[4-(N′,N″-dimethyl acryl-amide)-2-(1-H-tetrazol-5-yl)-phenyl]urea;
N-(4-Chloro-3-trifluoromethyl-phenyl)-N′-[4′-(piperidine-1-carbonyl)-3-(1H-tetrazol-5-yl)-biphenyl-4-yl]urea;
N-(4-Chloro-3-trifluoromethyl-phenyl)-N′-{4′-[carbonyl-(N″-methyl)-amino-acetic acid]-2-(1H-tetrazol-5-yl)-4-biphenyl}urea;
N-(4-Trifluoromethyl-3-chloro-phenyl)-N′-[2-(1H-tetrazol-5-yl)-4-(2-N,N-dimethylcarbamoyl-ethyl)-phenyl]urea;
N-(4-Fluoro-3-trifluoromethyl-phenyl)-N′-[4-fluoro-2-(1H-tetrazol-5-yl)-phenyl]-urea; or
N-(2-Naphthyl)-N′-[4-bromo-2-(1H-tetrazol-5-yl)phenyl]urea;
or a pharmaceutically acceptable salt thereof.

23. The method according to claim 1, wherein the diphenyl urea derivative is represented by Formula VII 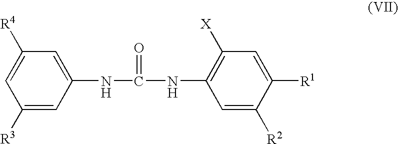
or a pharmaceutically acceptable salt thereof, wherein
X, R¹ and R² are as defined in claim 1, and
R³ represents hydrogen, alkyl, halo, haloalkyl, haloalkoxy, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy or phenyl; or phenyl substituted with haloalkyl.

24. The method according to claim 23, wherein
X represents hydroxy or carboxy;
R¹ represents hydrogen, halo, hydroxy, alkoxy, nitro, amino, N-phenyl-amino, N-benzoyl-amino, alkyl-carbonyl-amino, N-benzoyl-amino, alkoxy-carbonyl, phenyl, naphthyl, pyridyl, furyl or thienyl; or
R¹ represents phenyl substituted with halo, haloalkyl, nitro, carboxy, alkoxy-carbonyl, amino-carbonyl (carbamoyl), N,N-dialkyl-amino-carbonyl, N-phenyl-amino-carbonyl, anilino-carbonyl, amino-carbonyl-N-alkyl-piperazine, N,N-dialkyl-sulfamoyl or sulfonamido-N-alkyl-piperazinium chloride; and
R² represents hydrogen, halo, alkoxy, alkoxy-carbonyl, nitro, halophenyl, haloalkyl-phenyl or haloalkoxy-phenyl;
R³ represents hydrogen, alkyl, halo, haloalkyl, haloalkoxy, nitro, hydroxy, alkoxy, carboxy, alkyl-carbonyl, alkoxy-carbonyl, amino-carbonyl, benzoyl, acetyl, phenyl, or pyridyl; or phenyl substituted with alkyl, halo or haloalkyl; and
R⁴ represents hydrogen, alkyl, halo, haloalkyl, nitro, alkoxy or phenyl; or phenyl substituted with haloalkyl.

25. The method according to claim 24, wherein
X represents hydroxy or carboxy;
R¹ represents hydrogen, halo, hydroxy, alkoxy, nitro, alkoxy-carbonyl or N-phenyl-amino;
R² represents hydrogen, halo, alkoxy, alkoxy-carbonyl or nitro;
R³ represents alkyl, haloalkyl, haloalkoxy, nitro, hydroxy, carboxy, alkoxy-carbonyl, amino-carbonyl or benzoyl; and
R⁴ represents hydrogen.

26. The method according to claim 25, wherein the diphenyl urea derivative is
N-(3-(Trifluoromethyl)phenyl)-N′-(2-hydroxyphenyl)urea;
N-(3-(Trifluoromethyl)phenyl)-N′-(2-hydroxy-4-methoxyphenyl)urea;
N-(3-(Trifluoromethyl)phenyl)-N′-(2-hydroxy-4-methoxycarbonylphenyl)urea;
N-(3-(Trifluoromethyl)phenyl)-N′-(2-hydroxy-4-chlorophenyl)urea;
N-(3-(Trifluoromethyl)phenyl)-N′-(2-hydroxy-4-nitrophenyl)urea;
N-(3-(Trifluoromethyl)phenyl)-N′-(2-hydroxy-4-(phenylamino)phenyl)urea;
N-(3-(Trifluoromethyl)phenyl)-N′-(2,4-dihydroxyphenyl)urea;
N-(3-(Trifluoromethyl)phenyl)-N′-(2-hydroxy-4-methoxycarbonyl-5-chlorophenyl)urea;
N-(3-(Trifluoromethoxy)phenyl)-N′-(2-hydroxy-5-chlorophenyl)urea;
N-(3-(Trifluoromethyl)phenyl)-N′-(2-hydroxy-5-methoxycarbonylphenyl)urea;
N-(3-(Trifluoromethyl)phenyl)-N′-(2-hydroxy-5-nitrophenyl)urea;
N-(3-(Trifluoromethyl)phenyl)-N′-(2-hydroxy-5-chlorophenyl)urea;
N-(3-(Trifluoromethyl)phenyl)-N′-(2-hydroxy-5-methoxyphenyl)urea;
N-(3-(Trifluoromethyl)phenyl)-N′-(2-hydroxy-5-nitrophenyl)urea;
N-(3-Benzoylphenyl)-N′-(2-hydroxy-5-chlorophenyl)urea;
N-(3-Carbamoylphenyl)-N′-(2-hydroxy-5-chlorophenyl)urea;
N-(3-Carboxyphenyl)-N′-(2-hydroxy-5-chlorophenyl)urea;
N-(3-Hydroxyphenyl)-N′-(2-hydroxy-5-chlorophenyl)urea;
N-(3-Methoxycarbonylphenyl)-N′-(2-hydroxy-5-chlorophenyl)urea;
N-(3-Methylphenyl)-N′-(2-hydroxy-5-chlorophenyl)urea; or
N-(3-Nitrophenyl)-N′-(2-hydroxy-5-chlorophenyl)urea;
or a pharmaceutically acceptable salt thereof.

27. The method according to claim 23, wherein
X represents carboxy;
R¹ represents halo or phenyl;
R² represents hydrogen;
R³ represents haloalkyl; and
R⁴ represents hydrogen or haloalkyl.

28. The method according to claim 27, wherein the diphenyl urea derivative is
N-(3-Trifluoromethylphenyl)-N′-(2-carboxy-4-bromophenyl)urea;
N-(3-Trifluoromethylphenyl)-N′-(2-carboxy-4-chlorophenyl)urea;
N-(3-Trifluoromethylphenyl)-N′-(2-carboxy-4-fluorophenyl)urea;
N-(3-Trifluoromethylphenyl)-N′-(2-carboxy-4-trifluoromethylphenyl)urea;
N-(3-Trifluoromethylphenyl)-N′-(2-carboxy-4-biphenyl)urea; or
N-(3,5-Bis-trifluoromethylphenyl)-N′-(2-carboxy-4-biphenyl)urea;
or a pharmaceutically acceptable salt thereof.

29. The method according to claim 23, wherein
X represents a tetrazolyl group, an oxadiazolyl group or a triazolyl group;
R¹ represents hydrogen, halo, hydroxy, alkoxy, nitro, amino, N-phenyl-amino, alkyl-carbonyl-amino, N-benzoyl-amino, N,N-dialkyl acryl-amide, 2-N,N-dialkyl-carbamoyl-ethyl, alkyl-carbonyl, alkoxy-carbonyl, phenyl, naphthyl, pyridyl, furyl or thienyl; or
R¹ represents phenyl substituted with halo, haloalkyl, haloalkoxy, nitro, carboxy, alkoxy-carbonyl, amino-carbonyl (carbamoyl), N,N-dialkyl-amino-carbonyl, N-phenyl-amino-carbonyl, anilino-carbonyl, N-acetic acid-amino-carbonyl, N-alkyl-N-acetic acid-amino-carbonyl, carbamoyl-N-alkyl-piperazine, N,N-dialkyl-sulfamoyl or sulfonamido-N-alkyl-piperazinium chloride; and
R² represents hydrogen, halo, alkoxy, alkoxy-carbonyl, nitro, halophenyl, haloalkyl-phenyl or haloalkoxy-phenyl;
R³ represents hydrogen, alkyl, halo, haloalkyl, nitro, hydroxy, alkoxy, carboxy, alkyl-carbonyl, alkoxy-carbonyl, amino-carbonyl, benzoyl, acetyl, phenyl or pyridyl; or
R³ represents phenyl substituted with alkyl, halo or haloalkyl; and
R⁴ represents hydrogen, alkyl, halo, haloalkyl, nitro, alkoxy, phenyl or phenyl substituted with haloalkyl.

30. The method according to claim 29, wherein
X represents tetrazolyl;
R¹ represents hydrogen, halo, nitro, amino, alkyl-carbonyl, alkyl-carbonyl-amino, N-benzoyl-amino, phenyl, naphthyl, pyridyl, furyl or thienyl; or
R¹ represents phenyl substituted with halo, haloalkyl, nitro, carboxy, alkoxy-carbonyl, amino-carbonyl, N,N-dialkyl-amino-carbonyl, N-phenyl-amino-carbonyl, N,N-dialkyl-sulfamoyl, N-phenyl-amino-carbonyl, sulfonamido-N-alkyl-piperazinium chloride, carbamoyl-N-alkyl-piperazine, anilino-carbonyl; and
R² represents hydrogen;
R³ represents alkyl, halo, haloalkyl, nitro, alkoxy, alkyl-carbonyl, phenyl or pyridyl; and
R⁴ represents hydrogen.

31. The method according to claim 30, wherein the diphenyl urea derivative is
N-3-Trifluoromethylphenyl-N′-2-(1H-tetrazol-5-yl)phenyl urea;
N-3-Trifluoromethylphenyl-N′-4-nitro-2-(1H-tetrazol-5-yl)phenyl urea;
N-3-Trifluoromethylphenyl-N′-4-(1-naphthyl)-2-(1H-tetrazol-5-yl)phenyl urea;
N-3-Trifluoromethylphenyl-N′-4-(2-naphthyl)-2-(1H-tetrazol-5-yl)phenyl urea;
N-3-Trifluoromethylphenyl-N′-4-(3-pyridyl)-2-(1H-tetrazol-5-yl)phenyl urea;
N-3-Trifluoromethylphenyl-N′-4-(4-trifluoromethylphenyl)-2-(1H-tetrazol-5-yl)phenyl urea;
N-3-Trifluoromethylphenyl-N′-4-(3-furyl)-2-(1H-tetrazol-5-yl)phenyl urea;
N-3-Trifluoromethylphenyl-N′-4-(3-thienyl)-2-(1H-tetrazol-5-yl)phenyl urea;
N-3-Trifluoromethylphenyl-N′-4-(3-nitrophenyl)-2-(1H-tetrazol-5-yl)phenyl urea;
N-3-Trifluoromethylphenyl-N′-4-(4-ethoxycarbonylphenyl)-2-(1H-tetrazol-5-yl)phenyl urea;
N-3-Trifluoromethylphenyl-N′-4-(4-aminocarbonylphenyl)-2-(1H-tetrazol-5-yl)phenyl urea;
N-3-Trifluoromethylphenyl-N′-4-(4-diethylaminocarbonylphenyl)-2-(1H-tetrazol-5-yl)phenyl urea;
N-3-Trifluoromethylphenyl-N′-4-(4-phenylaminocarbonylphenyl)-2-(1H-tetrazol-5-yl)phenyl urea;
N-3-Trifluoromethylphenyl-N′-4-(4-benzoylamino-phenyl)-2-(1H-tetrazol-5-yl)phenyl urea;
N-3-Trifluoromethylphenyl-N′-4-amino-2-(1H-tetrazol-5-yl)phenyl urea;
N-3-Trifluoromethylphenyl-N′-4-acetylamino-2-(1H-tetrazol-5-yl)phenyl urea;
N-3-Trifluoromethylphenyl-N′-4-benzoylamino-2-(1H-tetrazol-5-yl)phenyl urea;
N-3-Trifluoromethylphenyl-N′-4-(4-carboxyphenyl)-2-(1H-tetrazol-5-yl)phenyl urea;
N-3-Trifluoromethylphenyl-N′-4-(4-anilinocarbonylphenyl)-2-(1H-tetrazol-5-yl)phenyl urea;
N-3-Biphenylyl-N′-2-(1H-tetrazol-5-yl)phenyl urea;
N-3-Bromophenyl-N′-4-bromo-2-(1H-tetrazol-5-yl)phenyl urea;
N-3-Acetylphenyl-N′-2-(1H-tetrazol-5-yl)phenyl urea;
N-3-Biphenylyl-N′-4-bromo-2-(1H-tetrazol-5-yl)phenyl urea; or
N-3-(3-Pyridyl)phenyl-N′-4-bromo-2-(1H-tetrazol-5-yl)phenyl urea;
N-(3-Bromophenyl)-N′-(2-(1H-tetrazol-5-yl)-4-biphenyl)urea;
N-(3-Trifluoromethylphenyl)-N′-(2-(1H-tetrazol-5-yl)-4-biphenyl)urea;
N-(3-Trifluoromethylphenyl)-N′-(4′-(N,N-dimethylsulfamoyl)-2-(1H-tetrazol-5-yl)-4-biphenyl)urea;
N-(3-Bromophenyl)-N′-(4′-(N,N-dimethylsulfamoyl)-2-(1H-tetrazol-5-yl)-4-biphenyl)urea;
N-(3-Bromophenyl)-N′-(4′-(N,N-dimethylcarbamoyl)-2-(1H-tetrazol-5-yl)-4-biphenyl)urea;
N-(3-Trifluoromethylphenyl)-N′-(4-amino-2-(1H-tetrazol-5-yl)phenyl)urea;
N-(3-Trifluoromethylphenyl)-N′-(4-acetylamino-2-(1H-tetrazol-5-yl)phenyl)urea;
N-(3-Trifluoromethylphenyl)-N′-(4′-carbamoyl-2-(1H-tetrazol-5-yl)-4-biphenyl)urea;
N-(3-Trifluoromethylphenyl)-N′-(4′-(N,N-dimethylcarbamoyl)-2-(1H-tetrazol-5-yl)-4-biphenyl)urea;
N-(3-Trifluoromethylphenyl)-N′-(4′-carboxy-2-(1H-tetrazol-5-yl)-4-biphenyl)urea;
N-(3-Trifluoromethylphenyl)-N′-(4′-(N-phenylcarbamoyl)-2-(1H-tetrazol-5-yl)-4-biphenyl)urea;
N-(3-Biphenyl)-N′-(2-(1H-tetrazol-5-yl)phenyl)urea;
N-(3-Acetylphenyl)-N′-(2-(1H-tetrazol-5-yl)phenyl)urea;
N-(3-Biphenyl)-N′-(4-bromo-2-(1H-tetrazol-5-yl)phenyl)urea;
N-(3-(3-Pyridyl)phenyl)-N′-(4-bromo-2-(1H-tetrazol-5-yl)phenyl)urea;
N-(3-Bromophenyl)-N′-(4-bromo-2-(1H-tetrazol-5-yl)phenyl)urea;
N-(3-Trifluoromethylphenyl)-N′-4-(4-benzoylcarbonylphenyl)-2-(1H-tetrazol-5-yl)phenyl urea;
N-(3-Bromophenyl)-N′-[3′-nitro-2-(1H-tetrazol-5-yl)biphenyl]urea;
N-(3-Bromophenyl)-N′-[4′-(sulfonamido-N′-methylpiperazinium chloride)-2-(1H-tetrazol-5-yl)-4-biphenyl]urea;
N-(3-Bromophenyl)-N′-[4′-carbamoyl-N′-methylpiperazine)-2-(1H-tetrazol-5-yl)-4′-biphenyl]urea;
N-(3-Methoxyphenyl)-N′-[4-bromo-2-(1H-tetrazol-5-yl)phenyl]urea;
N-(3-Chlorophenyl)-N′-[4-bromo-2-(1H-tetrazol-5-yl)phenyl]urea;
N-(3-Methylphenyl)-N′-[4-bromo-2-(1H-tetrazol-5-yl)phenyl]urea;
N-(3-Fluorophenyl)-N′-[4-bromo-2-(1H-tetrazol-yl)phenyl]urea;
N-(3-Nitrophenyl)-N′-[4-bromo-2-(1H-tetrazol-5-yl)phenyl]urea;
N-(3-Acetylphenyl)-N′-[4-bromo-2-(1H-tetrazol-5-yl)phenyl]urea;
N-(3-Trifluoromethylphenyl)-N′-[4-fluoro-2-(1H-tetrazol-yl)phenyl]urea;
N-(3-Trifluoromethylphenyl)-N′-[4-bromo-2-(1H-tetrazol-yl)phenyl]urea;
N-(3-Trifluoromethylphenyl)-N′-[3-(1H-tetrazol-5-yl)-4′-trifluoromethyl-biphenyl-4-yl]-urea;
N-(3-Trifluoromethylphenyl)-N′-[4′-chloro-3-(1H-tetrazol-5-yl)-biphenyl-4-yl]-urea;
N-(3-Trifluoromethylphenyl)-N′-[4′-fluoro-3-(1H-tetrazol-5-yl)-biphenyl-5-yl]-urea;
N-(3-Bromophenyl)-N′-[3-(1H-tetrazol-5-yl)-4′-trifluoromethyl-biphenyl-4-yl]-urea;
or a pharmaceutically acceptable salt thereof.

32. The method according to claim 29, wherein
X represents tetrazolyl;
R¹ represents halo, N,N-dialkyl acryl-amide, N,N-dialkyl-amino-carbonyl-alkyl, phenyl, or
R¹ represents phenyl substituted in position 3 or 4 with halo, haloalkyl, haloalkoxy, amino-carbonyl, N,N-dialkyl-sulfamoyl, N,N-dialkyl-amino-carbonyl, N-acetic acid-amino-carbonyl, N-alkyl-N-acetic acid-amino-carbonyl or anilino-carbonyl; and
R² represents hydrogen;
R³ represents alkyl, halo or haloalkyl; and
R⁴ represents alkyl, halo or haloalkyl.

33. The method according to claim 32, wherein the diphenyl urea derivative is
N-(3,5-Dichlorophenyl)-N′-[4-bromo-2-(1H-tetrazol-5-yl)phenyl]urea;
N-(3,5-Bis-trifluoromethylphenyl)-N′-[4-bromo-2-(1H-tetrazol-5-yl)phenyl]urea;
N-(3,5-Bis-trifluoromethylphenyl)-N′-[4-chloro-2-(1H-tetrazol-5-yl)phenyl]urea;
N-(3,5-Dichlorophenyl)-N′-[4-chloro-2-(1H-tetrazol-5-yl)phenyl]urea;
N-(3,5-Difluorophenyl)-N′-[4′-chloro-2-(1H-tetrazol-5-yl)phenyl]urea;
N-(3,5-Dimethylphenyl)-N′-[4-bromo-2-(1H-tetrazol-5-yl)phenyl]urea;
N-(3,5-Dichlorophenyl)-N′-[4′-chloro-3-(1H-tetrazol-5-yl)-biphenyl-4-yl]-urea;
N-(3,5-Difluorophenyl)-N′-[4′-chloro-3-(1H-tetrazol-5-yl)-biphenyl-4-yl]-urea;
N-(3,5-Dichlorophenyl)-N′-[3-(1H-tetrazol-5-yl)-4′-trifluoromethyl-biphenyl-4-yl]-urea;
N-(3,5-Difluorophenyl)-N′-[3-(1H-tetrazol-5-yl)-4′-trifluoromethyl-biphenyl-4-yl]-urea;
N-(3,5-Dichlorophenyl)-N′-[4′-fluoro-3-(1H-tetrazol-5-yl)-biphenyl-4-yl]-urea;
N-(3,5-Difluorophenyl)-N′-[4′-fluoro-2-(1H-tetrazol-5-yl)-biphenyl-4-yl]-urea;
N-(3,5-Bis-trifluoromethylphenyl)-N′-[3-(1H-tetrazol-5-yl)-4′-trifluoromethyl-biphenyl-4-yl]-urea;
N-(3,5-Bis-trifluoromethylphenyl)-N′-[4′-chloro-3-(1H-tetrazol-5-yl)-biphenyl-4-yl]-urea;
N-(3,5-Bis-trifluoromethylphenyl)-N′-[4′-fluoro-3-(1H-tetrazol-5-yl)-biphenyl-4-yl]-urea;
N-(3,5-Dichlorophenyl)-N′-[4′-methoxy-3-(1H-tetrazol-5-yl)-biphenyl-4-yl]-urea;
N-(3,5-Difluorophenyl)-N′-[3-(1H-tetrazol-5-yl)-4′-trifluoromethoxy-biphenyl-4-yl]-urea;
N-(3,5-Dichlorophenyl)-N′-[3-(1H-tetrazol-5-yl)-4′-trifluoromethoxy-biphenyl-4-yl]-urea;
N-(3,5-Bis-trifluoromethylphenyl)-N′-[3-(1H-tetrazol-5-yl)-4′-trifluoromethoxy-biphenyl-4-yl]-urea;
N-(3,5-Difluorophenyl)-N′-[3-(1H-tetrazol-5-yl)-3′-trifluoromethyl-biphenyl-4-yl]-urea;
N-(3,5-Dichlorophenyl)-N′-[3-(1H-tetrazol-5-yl)-3′-trifluoromethyl-biphenyl-4-yl]-urea;
N-(3,5-Bis-trifluoromethylphenyl)-N′-[3-(1H-tetrazol-5-yl)-3′-trifluoromethyl-biphenyl-4-yl]-urea;
N-(3,5-Difluorophenyl)-N′-[4′-methoxy-3-(1H-tetrazol-5-yl)-biphenyl-4-yl]-urea;
N-(3,5-Bis-trifluoromethylphenyl)-N′-[4′-methoxy-3-(1H-tetrazol-5-yl)-biphenyl-4-yl]-urea;
N-(3,5-Bis-trifluoromethylphenyl)-N′-(4′-(N,N-dimethylsulfamoyl)-2-(1H-tetrazol-5-yl)-4-biphenyl)urea;
N-(3,5-Difluorophenyl)-N′-(4′-carbamoyl-2-(1H-tetrazol-5-yl)-4-biphenyl)urea;
N-(3,5-Dichlorophenyl)-N′-(4′-(N,N-dimethylcarbamoyl)-2-(1H-tetrazol-5-yl)-4-biphenyl)urea;
N-(3,5-Bis-trifluoromethylphenyl)-N′-(4′-(N,N-dimethylcarbamoyl)-2-(1H-tetrazol-5-yl)-4-biphenyl)urea;
N-(3,5-Bis-trifluoromethylphenyl)-N′-(4′-carbamoyl-2-(1H-tetrazol-5-yl)-4-biphenyl)urea;
N-(3,5-Dichlorophenyl)-N′-(4′-(N,N-dimethylsulfamoyl)-2-(1H-tetrazol-5-yl)-4-biphenyl)urea;
N-(3,5-Difluorophenyl)-N′-(4′-(N,N-dimethylsulfamoyl)-2-(1H-tetrazol-5-yl)-4-biphenyl)urea;
N-(3-Trifluoromethyl-5-fluoro-phenyl)-N′-(4′-(N,N-dimethylsulfamoyl)-2-(1H-tetrazol-5-yl)-4-biphenyl)urea;
N-(3,5-Bis-trifluoromethylphenyl)-N′-4-(4-anilinocarbonylphenyl)-2-(1H-tetrazol-5-yl)phenyl urea;
N-(3,5-Bis-trifluoromethyl-phenyl)-N′-{4′-[carbonyl-amino-acetic acid]-2-(1H-tetrazol-5-yl)-4-biphenyl}urea;
N-(3,5-Difluorophenyl)-N′-{4′-[carbonyl-(N″-methyl)-amino-acetic acid]-2-(1H-tetrazol-5-yl)-4-biphenyl}urea;
N-(3,5-Bis-trifluoromethyl-phenyl)-N′-{4′-[carbonyl-(N″-methyl)-amino-acetic acid]-2-(1H-tetrazol-5-yl)-4-biphenyl}urea;
N-(3,5-Dichloro-phenyl)-N′-[4-(N″,N″-dimethyl acryl-amide)-2-(1-H-tetrazol-5-yl)-phenyl]urea;
N-(3,5-Dichloro-phenyl)-N′-[2-(1H-tetrazol-5-yl)-4-(2-N,N-dimethyl-carbamoyl-ethyl)-phenyl]urea;
N-(3,5-Bis-trifluoromethylphenyl)-N′-[2-(1H-tetrazol-5-yl)-4-(2-N,N-dimethyl-carbamoyl-ethyl)-phenyl]urea;
or a pharmaceutically acceptable salt thereof.

34. The method according to claim 32, wherein the diphenyl urea derivative is
N-(3,5-Dichlorophenyl)-N′-[3-(1H-tetrazol-5-yl)-3′-trifluoromethyl-biphenyl-4-yl]-urea;
or a pharmaceutically acceptable salt thereof.

35. The method according to claim 29, wherein
X represents an oxadiazolyl group;
R¹ represents hydrogen;
R² represents hydrogen;
R³ represents haloalkyl; and
R⁴ represents hydrogen.

36. The method according to claim 35, wherein the diphenyl urea derivative is
N-(3-Trifluoromethylphenyl)-N′-2-(2-oxo-3H-1,3,4-oxadiazol-5-yl)phenyl urea; or
N-(3-Trifluoromethylphenyl)-N′-[2-(5-oxo-4,5-dihydro-[1,2,4]oxadiazol-3-yl)-4-(4′-N,N-dimethyl-carbamoyl)-biphenyl]urea;
or a pharmaceutically acceptable salt thereof.

37. The use method according to claim 29, wherein
X represents 4-hydroxy-1,2,4-triazol-3-yl or 3-oxo-1,2-dihydro-1,2,4-triazol-1-yl;
R¹ represents hydrogen or phenyl;
R² represents hydrogen;
R³ represents trifluoromethyl; and
R⁴ represents hydrogen.

38. The method according to claim 37, wherein the diphenyl urea derivative is
N-3-Trifluoromethylphenyl-N′-2-(4-hydroxy-1,2,4-triazol-3-yl)phenyl urea;
N-3-Trifluoromethylphenyl-N′-2-(3-oxo-1,2-dihydro-1,2,4-triazol-1-yl)phenyl urea; or
N-3-Trifluoromethylphenyl-N′-4-biphenylyl-2-(3-oxo-1,2-dihydro-1,2,4-triazol-1-yl)phenyl urea;
or a pharmaceutically acceptable salt thereof.

39. (canceled)

40. The method according to claim 1, wherein the disease, disorder or condition is an obstructive or inflammatory airway disease.

41. The method according to claim 40, wherein the obstructive or inflammatory airway disease is an acute or chronic infectious pulmonary disease, an airway hyperreactivity, pneumoconiosis, aluminosis, anthracosis, asbestosis, chalicosis, ptilosis, siderosis, silicosis, tabacosis, byssinosis, sarcoidosis, berylliosis, a chronic obstructive pulmonary disease (COPD), bronchitis, chronic bronchitis, wheezy bronchitis, pulmonary emphysema, acute respiratory distress syndrome (ARDS) and acute lung injury (ALI), excerbation of airways hyperreactivity or cystic fibrosis, pulmonary fibrosis, Acute Respiratory Distress Syndrome (ARDS), pulmonary hypertension, inflammatory lung diseases, acute or chronic respiratory infectious diseases.

42. (canceled)

43. (canceled)

44. (canceled)

45. (canceled)
